(19) United States
(12) Patent Application Publication
(10) Pub. No.: US 2024/0304304 A1
(43) Pub. Date: Sep. 12, 2024

(54) METHODS AND SYSTEMS FOR DETERMINING A COMPATIBLE SUBSTANCE

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 18/661,324

(22) Filed: May 10, 2024

Related U.S. Application Data
(63) Continuation-in-part of application No. 16/530,329, filed on Aug. 2, 2019, now Pat. No. 11,984,199.

(51) Int. Cl. G16H 20/60; G06T 7/00; G06T 13/40; G16H 50/30

(52) U.S. Cl. CPC G16H 20/60; G06T 7/0012; G06T 13/40; G16H 50/30; G06T 2207/20081; G06T 2207/20084

(57) ABSTRACT

Described herein are systems and methods for determining a compatible substance. In some embodiments, a system may include a camera; a user interface; and a computing device configured to, using the camera, capture a first image; generate a first body measurement by training a body measurement machine learning model on a training dataset including a plurality of example images correlated to a plurality of example body measurements; and generating the first body measurement as a function of the first image using the trained body measurement machine learning model; determine a first compatible substance as a function of the first body measurement; and using the user interface, display the first compatible substance.

### CROSS-REFERENCE TO RELATED APPLICATIONS

This application is a continuation-in-part of Non-provisional application Ser. No. 16/530,329 filed on Aug. 2, 2019, and entitled “METHODS AND SYSTEMS FOR GENERATING COMPATIBLE SUBSTANCE INSTRUCTION SETS USING ARTIFICIAL INTELLIGENCE,” the entirety of which is incorporated herein by reference.

### FIELD OF THE INVENTION

The present invention generally relates to the field of artificial intelligence. In particular, the present invention is directed to methods and systems for determining a compatible substance.

### BACKGROUND

Accurate assessment of compatibility is challenging due to the vast magnitude of factors to be considered and analyzed. Incorrect assessment can prolong illness and detract from achieving a vibrant state. Accurate and informed analysis is of utmost importance when determining compatibility of substances.

### SUMMARY OF THE DISCLOSURE

In an aspect, a system for determining a compatible substance may include a camera; a user interface; and a computing device configured to, using the camera, capture a first image; generate a first body measurement by training a body measurement machine learning model on a training dataset including a plurality of example images correlated to a plurality of example body measurements; and generating the first body measurement as a function of the first image using the trained body measurement machine learning model; determine a first compatible substance as a function of the first body measurement; and using the user interface, display the first compatible substance.

In another aspect, a method of determining a compatible substance may include, using a camera and at least a processor, capturing a first image; using the at least a processor, generating a first body measurement by training a body measurement machine learning model on a training dataset including a plurality of example images correlated to a plurality of example body measurements; and generating the first body measurement as a function of the first image using the trained body measurement machine learning model; using the at least a processor, determining a first compatible substance as a function of the first body measurement; and using the user interface and the at least a processor, displaying the first compatible substance.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for determining a compatible substance;

FIG. 2 is a block diagram illustrating embodiments of data storage facilities for use in disclosed systems and methods;

FIG. 3 is a block diagram illustrating an exemplary embodiment of a classified biomarker database;

FIG. 4 is a block diagram illustrating an exemplary embodiment of an expert knowledge database;

FIG. 5 is a block diagram illustrating an exemplary embodiment of a compatible substance database;

FIG. 6 is a block diagram illustrating an exemplary embodiment of a training set database;

FIG. 7 is a block diagram illustrating an exemplary embodiment of a machine-learning model database;

FIG. 8 is a block diagram of an exemplary embodiment of a machine learning model;

FIG. 9 is a schematic diagram of an exemplary embodiment of a neural network;

FIG. 10 is a schematic diagram of an exemplary embodiment of a neural network node;

FIG. 11 is a block diagram illustrating an exemplary embodiment of a tissue sample analysis database;

FIG. 12 is a block diagram illustrating an exemplary embodiment of a compatible substance index value database;

FIG. 13 is a block diagram illustrating an exemplary embodiment of a compatible substance classification database;

FIG. 14 is a block diagram illustrating an exemplary embodiment of a user database;

FIG. 15 is a flow diagram illustrating an exemplary embodiment of a method of generating a compatible substance instruction set using artificial intelligence;

FIG. 16 is a flow diagram illustrating an exemplary embodiment of a method of determining a compatible substance; and

FIG. 17 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for determining a compatible substance. In an embodiment, at least a biomarker datum is received. In an embodiment, at least a biomarker datum may include a tissue sample or an analysis of a bodily fluid. At least a server categorizes the at least a biomarker datum to produce at least a classified biomarker datum. At least a classified biomarker datum may be classified as a function of a dimension of the human body. At least a server receives training data and selects at least a first machine-learning model as a function of the training data. The at least a server generates at least a compatible substance instruction set containing at least a recommended compatible substance as a function of the at least a classified biomarker datum, the training data and the at least a first machine-learning model.

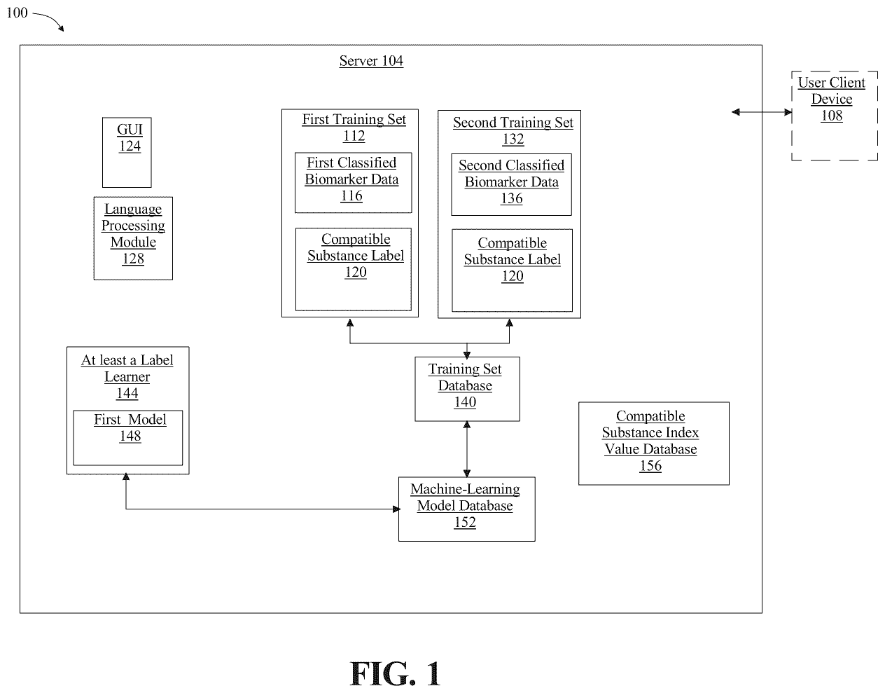

Turning now to FIG. 1, a system 100 for generating a compatible substance instruction set using artificial intelligence is illustrated. System 100 includes at least a server 104. At least a server 104 may include any computing device as described herein, including without limitation a microcontroller, microprocessor, digital signal processor (DSP) and/or system on a chip (SoC) as described herein. At least a server 104 may be housed with, may be incorporated in, or may incorporate one or more sensors of at least a sensor. Computing device may include, be included in, and/or communicate with a mobile device such as a mobile telephone or smartphone. At least a server 104 may include a single computing device operating independently, or may include two or more computing device operating in concert, in parallel, sequentially or the like; two or more computing devices may be included together in a single computing device or in two or more computing devices. At least a server 104 with one or more additional devices as described below in further detail via a network interface device. Network interface device may be utilized for connecting a at least a server 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. At least a server 104 may include but is not limited to, for example, at least a server 104 or cluster of computing devices in a first location and a second computing device or cluster of computing devices in a second location. At least a server 104 may include one or more computing devices dedicated to data storage, security, distribution of traffic for load balancing, and the like. At least a server 104 may distribute one or more computing tasks as described below across a plurality of computing devices of computing device, which may operate in parallel, in series, redundantly, or in any other manner used for distribution of tasks or memory between computing devices. At least a server 104 may be implemented using a “shared nothing” architecture in which data is cached at the worker, in an embodiment, this may enable scalability of system 100 and/or computing device.

With continued reference to FIG. 1, at least a server 104 is configured to receive at least a biomarker datum wherein the at least a biomarker datum contains at least an element of body data correlated to at least a body dimension. Biomarker datum, as used herein includes any element and/or elements of physiological state data. At least a biomarker datum may include a physically extraction sample, where a “physically extracted sample” as used in this disclosure is a sample obtained by removing and analyzing tissue and/or fluid. Physically extracted sample may include without limitation a blood sample, a tissue sample, a buccal swab, a mucous sample, a stool sample, a hair sample, a fingernail sample, or the like. For example and without limitation, at least a biomarker datum may include a hair sample that has been analyzed for specific nutrients or a saliva sample that has been analyzed for specific hormone levels. At least an element of body data may include at least a datum of user test data containing at least a root system label. User test data may include any data describing results obtained from a physically extracted sample from a user. For example, user test data may include results describing a urinalysis of a user examining for the absence or presence of ketones. In yet another non-limiting example, user test data may include results from a user's salivary hormone levels or results from a stool analysis. Root system label may include any label indicating a particular root cause of a user's test result. Root system may be correlated to a body dimension and may include information correlating a test result to a given body dimension. For example, at least an element of user test data showing elevated thyroid stimulating hormone level (TSH) outside normal limits may contain a root system label that indicates Hashimoto's thyroiditis which may be correlated to microbiome body dimension. In yet another non-limiting example, at least an element of user test data showing lactulose accumulated in urine sample after lactulose and mannitol consumption may contain a root system label that indicates leaky gut which may be correlated to gut wall body dimension.

With continued reference to FIG. 1, at least a biomarker datum may be categorized as a function of a biomarker system classification. Biomarker system classification, as used herein, includes categories of biomarker datums having shared characteristics as related to a dimension of the human body. Dimension of the human body includes particular root cause pillars of disease. Dimension of the human body include epigenetics, gut wall, microbiome, nutrients, genetics, and metabolism. Correcting deficiencies found within specific dimensions of the human body may aid a user in achieving vibrant health and longevity. At least an element of data contained within biomarker datum is correlated to at least a body dimension. Correlated may include a shared trait and/or shared data element classified to a particular body dimension. For instance and without limitation, a biomarker datum containing at least an element of microbiome data including for example species of specific strains of bacteria within the gastrointestinal tract may be correlated to a body dimension such as microbiome. In yet another non-limiting example, a biomarker datum containing at least an element of a phenotype of a particular gene may be correlated to a body dimension such as genetics. In an embodiment, at least an element of data may be correlated to a plurality of body dimensions. For instance and without limitation, at least an element of body data such as a stool chemistry analysis may be correlated to a microbiome body dimension and a gut wall body dimension.

With continued reference to FIG. 1, epigenetic, as used herein, includes any biomarker describing changes to a genome that do not involve corresponding changes in nucleotide sequence. Epigenetic biomarker may include data describing any heritable phenotypic. Phenotype, as used herein, include any observable trait of a user including morphology, physical form, and structure. Phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Epigenetic biomarkers may include data describing one or more states of methylation of genetic material.

With continued reference to FIG. 1, gut wall, as used herein, includes the space surrounding the lumen of the gastrointestinal tract that is composed of four layers including the mucosa, submucosa, muscular layer, and serosa. The mucosa contains the gut epithelium that is composed of goblet cells that function to secrete mucus, which aids in lubricating the passage of food throughout the digestive tract. The goblet cells also aid in protecting the intestinal wall from destruction by digestive enzymes. The mucosa includes villi or folds of the mucosa located in the small intestine that increase the surface area of the intestine. The villi contain a lacteal, that is a vessel connected to the lymph system that aids in removal of lipids and tissue fluids. Villi may contain microvilli that increase the surface area over which absorption can take place. The large intestine lack villi and instead a flat surface containing goblet cells are present.

With continued reference to FIG. 1, gut wall includes the submucosa, which contains nerves, blood vessels, and elastic fibers containing collagen. Elastic fibers contained within the submucosa aid in stretching the gastrointestinal tract with increased capacity while also maintaining the shape of the intestine. Gut wall includes muscular layer which contains smooth muscle that aids in peristalsis and the movement of digested material out of and along the gut. Gut wall includes the serosa which is composed of connective tissue and coated in mucus to prevent friction damage from the intestine rubbing against other tissue. Mesenteries are also found in the serosa and suspend the intestine in the abdominal cavity to stop it from being disturbed when a person is physically active.

With continued reference to FIG. 1, gut wall biomarker may include data describing one or more test results including results of gut wall function, gut wall integrity, gut wall strength, gut wall absorption, gut wall permeability, intestinal absorption, gut wall barrier function, gut wall absorption of bacteria, gut wall malabsorption, gut wall gastrointestinal imbalances and the like.

With continued reference to FIG. 1, gut wall biomarker may include data describing blood test results of creatinine levels, lactulose levels, zonulin levels, and mannitol levels. Gut wall biomarker may include blood test results of specific gut wall biomarkers including d-lactate, endotoxin lipopolysaccharide (LPS) Gut wall biomarker may include data breath tests measuring lactulose, hydrogen, methane, lactose, and the like. Gut wall biomarker may include blood test results describing blood chemistry levels of albumin, bilirubin, complete blood count, electrolytes, minerals, sodium, potassium, calcium, glucose, blood clotting factors,

With continued reference to FIG. 1, gut wall biomarker may include stool test results describing presence or absence of parasites, firmicutes, bacteriodetes, absorption, inflammation, food sensitivities. Stool test results may describe presence, absence, and/or measurement of acetate, aerobic bacterial cultures, anerobic bacterial cultures, fecal short chain fatty acids, beta-glucuronidase, cholesterol, chymotrypsin, fecal color, cryptosporidium EIA, Entamoeba histolytica, fecal lactoferrin, Giardia lamblia EIA, long chain fatty acids, meat fibers and vegetable fibers, mucus, occult blood, parasite identification, phospholipids, propionate, putrefactive short chain fatty acids, total fecal fat, triglycerides, yeast culture, n-butyrate, pH and the like.

With continued reference to FIG. 1, gut wall biomarker may include stool test results describing presence, absence, and/or measurement of microorganisms including bacteria, archaea, fungi, protozoa, algae, viruses, parasites, worms, and the like. Stool test results may contain species such as Bifidobacterium species, Campylobacter species, Clostridium difficile, Cryptosporidium species, Cyclospora cayetanensis, Cryptosporidium EIA, Dientamoeba fragilis, Entamoeba histolytica, Escherichia coli, Entamoeba histolytica, Giardia, H. pylori, Candida albicans, Lactobacillus species, worms, macroscopic worms, mycology, protozoa, Shiga toxin E. coli, and the like.

With continued reference to FIG. 1, gut wall biomarker may include microscopic ova exam results, microscopic parasite exam results, protozoan polymerase chain reaction test results and the like. Gut wall biomarker may include enzyme-linked immunosorbent assay (ELISA) test results describing immunoglobulin G (Ig G) food antibody results, immunoglobulin E (Ig E) food antibody results, Ig E mold results, IgG spice and herb results. Gut wall biomarker may include measurements of calprotectin, eosinophil protein x (EPX), stool weight, pancreatic elastase, total urine volume, blood creatinine levels, blood lactulose levels, blood mannitol levels.

With continued reference to FIG. 1, gut wall biomarker may include data describing one or more procedures examining gut including for example colonoscopy, endoscopy, large and small molecule challenge and subsequent urinary recovery using large molecules such as lactulose, polyethylene glycol-3350, and small molecules such as mannitol, L-rhamnose, polyethyleneglycol-400. Gut wall biomarker may include data describing one or more images such as x-ray, MRI, CT scan, ultrasound, standard barium follow-through examination, barium enema, barium with contract, MRI fluoroscopy, positron emission tomography 9PET), diffusion-weighted MRI imaging, and the like.

With continued reference to FIG. 1, microbiome, as used herein, includes ecological community of commensal, symbiotic, and pathogenic microorganisms that reside on or within any of a number of human tissues and biofluids. For example, human tissues and biofluids may include the skin, mammary glands, placenta, seminal fluid, uterus, vagina, ovarian follicles, lung, saliva, oral mucosa, conjunctiva, biliary, and gastrointestinal tracts. Microbiome may include for example, bacteria, archaea, protists, fungi, and viruses. Microbiome may include commensal organisms that exist within a human being without causing harm or disease. Microbiome may include organisms that are not harmful but rather harm the human when they produce toxic metabolites such as trimethylamine. Microbiome may include pathogenic organisms that cause host damage through virulence factors such as producing toxic by-products. Microbiome may include populations of microbes such as bacteria and yeasts that may inhabit the skin and mucosal surfaces in various parts of the body. Bacteria may include for example Firmicutes species, Bacteroidetes species, Proteobacteria species, Verrumicrobia species, Actinobacteria species, Fusobacteria species, Cyanobacteria species and the like. Archaea may include methanogens such as Methanobrevibacter smithii and Methanosphaera stadtmanae. Fungi may include Candida species and Malassezia species. Viruses may include bacteriophages. Microbiome species may vary in different locations throughout the body. For example, the genitourinary system may contain a high prevalence of Lactobacillus species while the gastrointestinal tract may contain a high prevalence of Bifidobacterium species while the lung may contain a high prevalence of Streptococcus and Staphylococcus species.

With continued reference to FIG. 1, microbiome biomarker may include stool test results describing presence, absence, and/or measurement of microorganisms including bacteria, archaea, fungi, protozoa, algae, viruses, parasites, worms, and the like. Stool test results may contain species such as Akkermansia muciniphila, Anaerotruncus colihominis, bacteriology, Bacteroides vulgatus, Bacteroides-Prevotella, Barnesiella species, Bifidobacterium longum, Bifidobacterium species, Butyrivbrio crossotus, Clostridium species, Collinsella aerofaciens, fecal color, fecal consistency, Coprococcus eutactus, Desulfovibrio piger, Escherichia coli, Faecalibacterium prausnitzii, Fecal occult blood, Firmicutes to Bacteroidetes ratio, Fusobacterium species, Lactobacillus species, Methanobrevibacter smithii, yeast minimum inhibitory concentration, bacteria minimum inhibitory concentration, yeast mycology, fungi mycology, Odoribacter species, Oxalobacter formigenes, parasitology, Prevotella species, Pseudoflavonifractor species, Roseburia species, Ruminococcus species, Veillonella species and the like.

With continued reference to FIG. 1, microbiome biomarker may include stool tests results that identify all microorganisms living a user's gut including bacteria, viruses, archaea, yeast, fungi, parasites, and bacteriophages. Microbiome biomarker may include DNA and RNA sequences from live microorganisms that may impact a user's health. Microbiome biomarker may include high resolution of both species and strains of all microorganisms. Microbiome biomarker may include data describing current microbe activity. Microbiome biomarker may include expression of levels of active microbial gene functions. Microbiome biomarker may include descriptions of sources of disease causing microorganisms, such as viruses found in the gastrointestinal tract such as raspberry bushy swarf virus from consuming contaminated raspberries or Pepino mosaic virus from consuming contaminated tomatoes.

With continued reference to FIG. 1, microbiome biomarker may include blood test results that identify metabolites produced by microorganisms. Metabolites may include for example, indole-3-propionic acid, indole-3-lactic acid, indole-3-acetic acid, tryptophan, serotonin, kynurenine, total indoxyl sulfate, tyrosine, xanthine, 3-methylxanthine, uric acid, and the like.

With continued reference to FIG. 1, microbiome biomarker may include breath test results that identify certain strains of microorganisms that may be present in certain areas of a user's body. This may include for example, lactose intolerance breath tests, methane based breath tests, hydrogen based breath tests, fructose based breath tests. Helicobacter pylori breath test, fructose intolerance breath test, bacterial overgrowth syndrome breath tests and the like.

With continued reference to FIG. 1, microbiome biomarker may include urinary analysis for certain microbial strains present in urine. This may include for example, urinalysis that examines urine specific gravity, urine cytology, urine sodium, urine culture, urinary calcium, urinary hematuria, urinary glucose levels, urinary acidity, urinary protein, urinary nitrites, bilirubin, red blood cell urinalysis, and the like.

With continued reference to FIG. 1, nutrient as used herein, includes any substance required by the human body to function. Nutrients may include carbohydrates, protein, lipids, vitamins, minerals, antioxidants, fatty acids, amino acids, and the like. Nutrients may include for example vitamins such as thiamine, riboflavin, niacin, pantothenic acid, pyridoxine, biotin, folate, cobalamin, Vitamin C, Vitamin A, Vitamin D, Vitamin E, and Vitamin K. Nutrients may include for example minerals such as sodium, chloride, potassium, calcium, phosphorous, magnesium, sulfur, iron, zinc, iodine, selenium, copper, manganese, fluoride, chromium, molybdenum, nickel, aluminum, silicon, vanadium, arsenic, and boron.

With continued reference to FIG. 1, nutrients may include extracellular nutrients that are free floating in blood and exist outside of cells. Extracellular nutrients may be located in serum. Nutrients may include intracellular nutrients which may be absorbed by cells including white blood cells and red blood cells.

With continued reference to FIG. 1, nutrient biomarker may include blood test results that identify extracellular and intracellular levels of nutrients. Nutrient biomarker may include blood test results that identify serum, white blood cell, and red blood cell levels of nutrients. For example, nutrient biomarker may include serum, white blood cell, and red blood cell levels of micronutrients such as Vitamin A, Vitamin B1, Vitamin B2, Vitamin B3, Vitamin B6, Vitamin B12, Vitamin B5, Vitamin C, Vitamin D, Vitamin E, Vitamin K1, Vitamin K2, and folate.

With continued reference to FIG. 1, nutrient biomarker may include blood test results that identify serum, white blood cell and red blood cell levels of nutrients such as calcium, manganese, zinc, copper, chromium, iron, magnesium, copper to zinc ratio, choline, inositol, carnitine, methylmalonic acid (MMA), sodium, potassium, asparagine, glutamine, serine, coenzyme q10, cysteine, alpha lipoic acid, glutathione, selenium, eicosapentaenoic acid (EPA), docosahexaenoic acid (DHA), docosapentaenoic acid (DPA), total omega-3, lauric acid, arachidonic acid, oleic acid, total omega 6, and omega 3 index.

With continued reference to FIG. 1, nutrient biomarker may include salivary test results that identify levels of nutrients including any of the nutrients as described herein. Nutrient biomarker may include hair analysis of levels of nutrients including any of the nutrients as described herein.

With continued reference to FIG. 1, genetic as used herein, includes any inherited trait. Inherited traits may include genetic material contained with DNA including for example, nucleotides. Nucleotides include adenine (A), cytosine (C), guanine (G), and thymine (T). Genetic information may be contained within the specific sequence of an individual's nucleotides and sequence throughout a gene or DNA chain. Genetics may include how a particular genetic sequence may contribute to a tendency to develop a certain disease such as cancer or Alzheimer's disease.

With continued reference to FIG. 1, genetic biomarker may include blood tests, hair tests, skin tests, urine, amniotic fluid, buccal swabs and/or tissue test to identify a user's particular sequence of nucleotides, genes, chromosomes, and/or proteins. Genetic biomarker may include tests that example genetic changes that may lead to genetic disorders. Genetic biomarker may detect genetic changes such as deletion of genetic material or pieces of chromosomes that may cause Duchenne Muscular Dystrophy. Genetic biomarker may detect genetic changes such as insertion of genetic material into DNA or a gene such as the BRCA1 gene that is associated with an increased risk of breast and ovarian cancer due to insertion of 2 extra nucleotides. Genetic biomarker may include a genetic change such as a genetic substitution from a piece of genetic material that replaces another as seen with sickle cell anemia where one nucleotide is substituted for another. Genetic biomarker may detect a genetic change such as a duplication when extra genetic material is duplicated one or more times within a person's genome such as with Charcot-Marie Tooth disease type 1. Genetic biomarker may include a genetic change such as an amplification when there is more than a normal number of copies of a gene in a cell such as HER2 amplification in cancer cells. Genetic biomarker may include a genetic change such as a chromosomal translocation when pieces of chromosomes break off and reattach to another chromosome such as with the BCR-ABL1 gene sequence that is formed when pieces of chromosome 9 and chromosome 22 break off and switch places. Genetic biomarker may include a genetic change such as an inversion when one chromosome experiences two breaks and the middle piece is flipped or inverted before reattaching. Genetic biomarker may include a repeat such as when regions of DNA contain a sequence of nucleotides that repeat a number of times such as for example in Huntington's disease or Fragile X syndrome. Genetic biomarker may include a genetic change such as a trisomy when there are three chromosomes instead of the usual pair as seen with Down syndrome with a trisomy of chromosome 21, Edwards syndrome with a trisomy at chromosome 18 or Patau syndrome with a trisomy at chromosome 13. Genetic biomarker may include a genetic change such as monosomy such as when there is an absence of a chromosome instead of a pair, such as in Turner syndrome.

With continued reference to FIG. 1, genetic biomarker may include an analysis of COMT gene that is responsible for producing enzymes that metabolize neurotransmitters. Genetic biomarker may include an analysis of DRD2 gene that produces dopamine receptors in the brain. Genetic biomarker may include an analysis of ADRA2B gene that produces receptors for noradrenaline. Genetic biomarker may include an analysis of 5-HTTLPR gene that produces receptors for serotonin. Genetic biomarker may include an analysis of BDNF gene that produces brain derived neurotrophic factor. Genetic biomarker may include an analysis of 9p21 gene that is associated with cardiovascular disease risk. Genetic biomarker may include an analysis of APOE gene that is involved in the transportation of blood lipids such as cholesterol. Genetic biomarker may include an analysis of NOS3 gene that is involved in producing enzymes involved in regulating vaso-dilation and vaso-constriction of blood vessels.

With continued reference to FIG. 1, genetic biomarker may include ACE gene that is involved in producing enzymes that regulate blood pressure. Genetic biomarker may include SLCO1B1 gene that directs pharmaceutical compounds such as statins into cells. Genetic biomarker may include FUT2 gene that produces enzymes that aid in absorption of Vitamin B12 from digestive tract. Genetic biomarker may include MTHFR gene that is responsible for producing enzymes that aid in metabolism and utilization of Vitamin B9 or folate. Genetic biomarker may include SHMT1 gene that aids in production and utilization of Vitamin B9 or folate. Genetic biomarker may include MTRR gene that produces enzymes that aid in metabolism and utilization of Vitamin B12. Genetic biomarker may include MTR gene that produces enzymes that aid in metabolism and utilization of Vitamin B12. Genetic biomarker may include FTO gene that aids in feelings of satiety or fulness after eating. Genetic biomarker may include MC4R gene that aids in producing hunger cues and hunger triggers. Genetic biomarker may include APOA2 gene that directs body to produce ApoA2 thereby affecting absorption of saturated fats. Genetic biomarker may include UCP1 gene that aids in controlling metabolic rate and thermoregulation of body. Genetic biomarker may include TCF7L2 gene that regulates insulin secretion. Genetic biomarker may include AMY1 gene that aids in digestion of starchy foods. Genetic biomarker may include MCM6 gene that controls production of lactase enzyme that aids in digesting lactose found in dairy products. Genetic biomarker may include BCMO1 gene that aids in producing enzymes that aid in metabolism and activation of Vitamin A. Genetic biomarker may include SLC23A1 gene that produce and transport Vitamin C. Genetic biomarker may include CYP2R1 gene that produce enzymes involved in production and activation of Vitamin D. Genetic biomarker may include GC gene that produce and transport Vitamin D. Genetic biomarker may include CYP1A2 gene that aid in metabolism and elimination of caffeine. Genetic biomarker may include CYP17A1 gene that produce enzymes that convert progesterone into androgens such as androstenedione, androstendiol, dehydroepiandrosterone, and testosterone.

With continued reference to FIG. 1, genetic biomarker may include CYP19A1 gene that produce enzymes that convert androgens such as androstenedione and testosterone into estrogens including estradiol and estrone. Genetic biomarker may include SRD5A2 gene that aids in production of enzymes that convert testosterone into dihydrotestosterone. Genetic biomarker may include UFT2B17 gene that produces enzymes that metabolize testosterone and dihydrotestosterone. Genetic biomarker may include CYP1A1 gene that produces enzymes that metabolize estrogens into 2 hydroxy-estrogen. Genetic biomarker may include CYP1B1 gene that produces enzymes that metabolize estrogens into 4 hydroxy-estrogen. Genetic biomarker may include CYP3A4 gene that produces enzymes that metabolize estrogen into 16 hydroxy-estrogen. Genetic biomarker may include COMT gene that produces enzymes that metabolize 2 hydroxy-estrogen and 4 hydroxy-estrogen into methoxy estrogen. Genetic biomarker may include GSTT1 gene that produces enzymes that eliminate toxic by-products generated from metabolism of estrogens. Genetic biomarker may include GSTM1 gene that produces enzymes responsible for eliminating harmful by-products generated from metabolism of estrogens. Genetic biomarker may include GSTP1 gene that produces enzymes that eliminate harmful by-products generated from metabolism of estrogens. Genetic biomarker may include SOD2 gene that produces enzymes that eliminate oxidant by-products generated from metabolism of estrogens.

With continued reference to FIG. 1, metabolic, as used herein, includes any process that converts food and nutrition into energy. Metabolic may include biochemical processes that occur within the body. Metabolic biomarker may include blood tests, hair tests, skin tests, amniotic fluid, buccal swabs and/or tissue test to identify a user's metabolism. Metabolic biomarker may include blood tests that examine glucose levels, electrolytes, fluid balance, kidney function, and liver function. Metabolic biomarker may include blood tests that examine calcium levels, albumin, total protein, chloride levels, sodium levels, potassium levels, carbon dioxide levels, bicarbonate levels, blood urea nitrogen, creatinine, alkaline phosphatase, alanine amino transferase, aspartate amino transferase, bilirubin, and the like.

With continued reference to FIG. 1, metabolic biomarker may include blood, saliva, hair, urine, skin, and/or buccal swabs that examine levels of hormones within the body such as 11-hydroxy-androstereone, 11-hydroxy-etiocholanolone, 11-keto-androsterone, 11-keto-etiocholanolone, 16 alpha-hydroxyestrone, 2-hydroxyestrone, 4-hydroxyestrone, 4-methoxyestrone, androstanediol, androsterone, creatinine, DHEA, estradiol, estriol, estrone, etiocholanolone, pregnanediol, pregnanestriol, specific gravity, testosterone, tetrahydrocortisol, tetrahydrocrotisone, tetrahydrodeoxycortisol, allo-tetrahydrocortisol.

With continued reference to FIG. 1, metabolic biomarker may include metabolic rate tests such as breath tests that may analyze a user's resting metabolic rate or number of calories that a user's body burns each day rest. Metabolic biomarker may include one or more vital signs including blood pressure, breathing rate, pulse rate, temperature, and the like. Metabolic biomarker may include blood tests such as a lipid panel such as low density lipoprotein (LDL), high density lipoprotein (HDL), triglycerides, total cholesterol, ratios of lipid levels such as total cholesterol to HDL ratio, insulin sensitivity test, fasting glucose test, Hemoglobin A1C test, adipokines such as leptin and adiponectin, neuropeptides such as ghrelin, pro-inflammatory cytokines such as interleukin 6 or tumor necrosis factor alpha, anti-inflammatory cytokines such as interleukin 10, markers of antioxidant status such as oxidized low-density lipoprotein, uric acid, paraoxonase 1.

With continued reference to FIG. 1, at least a biomarker datum may include a tissue sample analysis correlated to at least a body dimension. Tissue sample as used herein, includes any material extracted from a human body including bodily fluids and tissue. Tissue sample may include for example, blood, urine, sputum, fecal, and solid tissue such as bone or muscle. Tissue sample analysis as used herein, includes any tissue sample analyzed by a laboratory or medical professional such as a medical doctor for examination. In an embodiment, tissue sample analysis may include comparisons of tissue sample examination as compared to reference ranges of normal values or normal findings. For example, tissue sample analysis may include a report identifying strains of bacteria located within a user's gut examined from a stool sample. In yet another non-limiting example, tissue sample analysis may include a report identifying hormone levels of a pre-menopausal female examined from a saliva sample. In yet another non-limiting example, tissue sample analysis may include reported results from a buccal swab that examined genetic mutations of particular genes. In yet another non-limiting example, tissue sample analysis may include a finger-prick blood test that may identify intracellular and extracellular levels of particular nutrients such as Vitamin D, Vitamin C, and Coenzyme Q10.

With continued reference to FIG. 1, a user client device 108 may include, without limitation, a display in communication with at least a server 104; display may include any display as described herein. A user client device 108 may include an additional computing device, such as a mobile device, laptop, desktop computer, or the like; as a non-limiting example, the user client device 108 may be a computer and/or workstation operated by a medical professional. Output may be displayed on at least a user client device 108 using an output graphical user interface 124, as described in more detail below. Transmission to a user client device 108 may include any of the transmission methodologies as described herein.

With continued reference to FIG. 1, at least a server is designed and configured to receive training data. Training data, as used herein, is data containing correlation that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name and/or a description of a medical condition or therapy may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below.

With continued reference to FIG. 1, at least a server 104 is configured to receive a first training set 112 including a plurality of first data entries, each first data entry of the first training set 112 including at least a first element of first classified biomarker data 116 and at least a correlated compatible substance label 120. At least a first element of first classified biomarker data 116 as used herein, includes any data indicative a person's physiological state that has been classified. Physiological state, as used herein, includes any information or data describing the current condition of a user's body. Classified as used herein, includes any classification system that relates to a dimension of the human body as described above in more detail. For instance and without limitation, biomarker data describing population of a species of bacteria found within the gastrointestinal tract may be classified as microbiome. Biomarker data may be classified into more than one dimension of the human body. For instance and without limitation, biomarker data describing population of a species of bacteria found within the gastrointestinal tract may be classified as microbiome and gut wall. In yet another non-limiting example, biomarker data describing a genetic single nucleotide polymorphism that increases a user's risk of developing diabetes mellitus, may be classified as genetics and metabolism. In an embodiment, first element of classified biomarker data may be received from at least a constitutional analysis. Constitutional analysis as used herein includes any medical procedure or test performed to detect, diagnose, monitor disease, disease processes, susceptibility, and treatment. Constitutional analysis may include any direct to consumer test that a consumer may perform without the need for a medical professional to order the test such as a medical doctor or nurse practitioner. In an embodiment, first element of classified biomarker data may be received from at least a tissue sample analysis. Tissue sample analysis may include any of the tissue sample analysis as described herein.

With continued reference to FIG. 1, a correlated compatible substance label 120 as used herein, includes any element of data identifying and/or describing any food substance that is compatible with a user. Food substance, as used herein, includes any substance consumed to provide nutritional support for an organism such as a human being. Food substance may be of plant or animal origin, and may contain essential nutrients such as carbohydrates, fats, proteins, vitamins, or minerals. Food substance may be categorized into categories based on type of food substance such as vegetables, fruits, grains, proteins, fats, herbs, spices, and other. Food substance may include individual foods such as banana, Brussel sprout, egg, endive, garlic, hazelnut and the like.

With continued reference to FIG. 1, correlated compatible substance label 120 may be associated with one or more elements of classified biomarker data. For example, a correlated compatible substance label 120 for garlic may be associated with one or more classified biomarker datums including for example genetic MTHFR gene mutation type C/T as well as the microbiome microbial strain of Candida Albicans found in a stool sample. In yet another non-limiting example, a correlated compatible substance label 120 for chicken breast may be associated with one or more classified biomarker datums including for example, nutrient blood sample showing low serum levels of Vitamin B3 as well as metabolic data reflecting elevated low density lipoprotein (LDL) levels. In yet another non-limiting example, a correlated compatible substance label 120 for mozzarella cheese may be associated with nutrient data showing low intracellular and extracellular calcium as well as genetic data showing a user has A/A variant of MCM6 gene which regulates production of lactase enzyme which is not impaired with the A/A variant.

With continued reference to FIG. 1, biomarker datum and correlated compatible substance label 120 may be stored in any suitable data and/or data type. For instance, and without limitation, correlated compatible substance label 120 may include textual data, such as numerical, character, and/or string data. Textual data may include a standardized name and/or code for a disease, disorder, or the like; codes may include diagnostic codes and/or diagnosis codes, which may include without limitation codes used in diagnosis classification systems such as The International Statistical Classification of Diseases and Related Health Problems (ICD). In general, there is no limitation on forms textual data or non-textual data used as at least a correlated compatible substance label 120 may take; persons skilled in the art, upon reviewing the entirety of this disclosure, will be aware of various forms which may be suitable for use as at least an advisory label consistently with this disclosure.

With continued reference to FIG. 1, correlated compatible substance label 120 may be stored as image data, such as for example an image of a particular food substance such as a photograph of a pear or an image of a steak. Image data may be stored in various forms including for example, joint photographic experts group (JPEG), exchangeable image file format (Exif), tagged image file format (TIFF), graphics interchange format (GIF), portable network graphics (PNG), netpbm format, portable bitmap (PBM), portable any map (PNM), high efficiency image file format (HEIF), still picture interchange file format (SPIFF), better portable graphics (BPG), drawn filed, enhanced compression wavelet (ECW), flexible image transport system (FITS), free lossless image format (FLIF), graphics environment manage (GEM), portable arbitrary map (PAM), personal computer exchange (PCX), progressive graphics file (PGF), gerber formats, 2 dimensional vector formats, 3 dimensional vector formats, compound formats including both pixel and vector data such as encapsulated postscript (EPS), portable document format (PDF), and stereo formats.

With continued reference to FIG. 1, in each first data element of first training set 112 at least a first element of first classified biomarker data 116 is correlated with a compatible substance label 120 where the first element of first classified biomarker data 116 is located in the same data element and/or portion of data element as the compatible substance label 120; for example, and without limitation, an element of classified biomarker data is correlated with a compatible substance label 120 where both element of classified biomarker data and compatible substance label 120 are contained within the same first data element of the first training set 112. As a further example, an element of classified biomarker data is correlated with a correlated compatible substance label 120 where both share a category label as described in further detail below, where each is within a certain distance of the other within an ordered collection of data in data element, or the like. Still further, an element of classified biomarker data may be correlated with a correlated compatible substance label 120 where the element of classified biomarker data and the correlated compatible substance label 120 share an origin, such as being data that was collected with regard to a single person or the like. In an embodiment, a first datum may be more closely correlated with a second datum in the same data element than with a third datum contained in the same data element; for instance, the first element and the second element may be closer to each other in an ordered set of data than either is to the third element, the first element and second element may be contained in the same subdivision and/or section of data while the third element is in a different subdivision and/or section of data, or the like. Persons skilled in the art, upon reviewing the entirety of this disclosure, will be aware of various forms and/or degrees of correlation between classified biomarker data and correlated compatible substance label 120 that may exist in first training set 112 and/or first data element consistently with this disclosure.

With continued reference to FIG. 1, at least a server 104 may be designed and configured to associate at least an element of classified biomarker data with at least a category from a list of significant categories of classified biomarker data. Significant categories of classified biomarker data may include labels and/or descriptors describing types of classified biomarker data that are identified as being of high relevance in identifying compatible substance label 120. As a non-limiting example, one or more categories may identify significant categories of classified biomarker data based on degree of diagnostic relevance to one or more impactful body dimensions and/or within one or more medical or public health fields. For instance, and without limitation, a particular set of biomarkers, test results, and/or biochemical information may be recognized in a given medical field as useful for identifying various conditions or associated food substances that may be compatible with a particular condition as well as associated ingredients and food substances that may not be compatible with a particular condition. As a non-limiting example, and without limitation, biomarker data describing disorders associated with vegetarian diets such as elevated fasting blood sugar levels may be useful in selecting compatible substance label 120 that include fruits, vegetables, grains, and dairy and that avoid fish or meat. As an additional example, biomarker data associated with dyslipidemia such as the presence of APOE 4 gene or mutations of APOA2 gene may be useful in selecting compatible substance label 120 that do not contain saturated fat such as coconut oil and palm oil. In a further non-limiting example, biomarker data describing disorders of AMY1 gene that produce enzymes that digest starchy foods may be useful in selecting compatible substance label 120 that are free of starches including for example nuts such as almonds and hazelnuts, and non-starchy vegetables such as artichokes, asparagus, bean sprouts, Brussel sprouts, broccoli, cabbage and the like. Persons skilled in the art, upon reviewing the entirety of this disclosure, will be aware of various additional categories of biological data that may be used consistently with this disclosure.

Still referring to FIG. 1, at least a server 104 may receive the list of significant categories according to any suitable process; for instance, and without limitation, at least a server 104 may receive the list of significant categories from at least an expert. In an embodiment, at least a server 104 may provide a graphical user interface 124, which may include without limitation a form or other graphical element having data entry fields, wherein one or more experts, including without limitation clinical and/or scientific experts, may enter information describing one or more categories of biomarker data that the experts consider to be significant or useful for detection of conditions; fields in graphical user interface 124 may provide options describing previously identified categories, which may include a comprehensive or near-comprehensive list of types of biomarker data detectable using known or recorded testing methods, for instance in “drop-down” lists, where experts may be able to select one or more entries to indicate their usefulness and/or significance in the opinion of the experts. Fields may include free-form entry fields such as text-entry fields where an expert may be able to type or otherwise enter text, enabling expert to propose or suggest categories not currently recorded. First graphical user interface 124 or the like may include fields corresponding to correlated compatible substance label 120, where experts may enter data describing compatible substance label 120 and/or categories of compatible substance label 120 the experts consider related to entered categories of biomarker data; for instance, such fields may include drop-down lists or other pre-populated data entry fields listing currently recorded compatible substance label 120, and which may be comprehensive, permitting each expert to select a compatible substance label 120 and/or a plurality of compatible substance label 120 the expert believes to be predicted and/or associated with each category of classified biomarker data selected by the expert. Fields for entry of compatible substance label 120 and/or categories of compatible substance label 120 may include free-form data entry fields such as text entry fields; as described above, examiners may enter data not presented in pre-populated data fields in the free-form data entry fields. Alternatively or additionally, fields for entry of compatible substance label 120 may enable an expert to select and/or enter information describing or linked to a category of compatible substance label 120 that the expert considers significant, where significance may indicate likely impact on longevity, mortality, quality of life, or the like as described in further detail below. First graphical user interface 124 may provide an expert with a field in which to indicate a reference to a document describing significant categories of biomarker data, relationships of such categories to compatible substance label 120, and/or significant categories of compatible substance label 120. Any data described above may alternatively or additionally be received from experts similarly organized in paper form, which may be captured and entered into data in a similar way, or in a textual form such as a portable document file (PDF) with examiner entries, or the like.

With continued reference to FIG. 1, data information describing significant categories of biomarker data, relationships of such categories to compatible substance label 120, and/or significant categories of compatible substance label 120 may alternatively or additionally be extracted from one or more documents using a language processing module 128. Language processing module 128 may include any hardware and/or software module. Language processing module 128 may be configured to extract, from the one or more documents, one or more words. One or more words may include, without limitation, strings of one or characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above, Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring to FIG. 1, language processing module 128 may compare extracted words to categories of biomarker data recorded by at least a server 104, and/or one or more categories of compatible substance label 120 recorded by at least a server 104; such data for comparison may be entered on at least a server 104 as described above using expert data inputs or the like. In an embodiment, one or more categories may be enumerated, to find total count of mentions in such documents. Alternatively or additionally, language processing module 128 may operate to produce a language processing model. Language processing model may include a program automatically generated by at least a server 104 and/or language processing module 128 to produce associations between one or more words extracted from at least a document and detect associations, including without limitation mathematical associations, between such words, and/or associations of extracted words with categories of classified biomarker data, relationships of such categories to compatible substance label 120, and/or categories of compatible substance label 120. Associations between language elements, where language elements include for purposes herein extracted words, categories of classified biomarker data, relationships of such categories to compatible substance label 120, and/or categories of compatible substance label 120 may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of classified biomarker data, a given relationship of such categories to compatible substance label 120, and/or a given category of compatible substance label 120. As a further example, statistical correlations and/or mathematical associations may include probabilistic formulas or relationships indicating a positive and/or negative association between at least an extracted word and/or a given category of classified biomarker data, a given relationship of such categories to compatible substance label 120, and/or a given category of compatible substance label 120; positive or negative indication may include an indication that a given document is or is not indicating a category of classified biomarker data, relationship of such category to compatible substance label 120, and/or category of compatible substance label 120 is or is not significant. For instance, and without limitation, a negative indication may be determined from a phrase such as “Bacteroides species were not found to alter carbohydrate metabolism,” whereas a positive indication may be determined from a phrase such as “Lactobacillus species were found to alter carbohydrate metabolism” as an illustrative example; whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory by at least a server 104, or the like.

Still referring to FIG. 1, language processing module 128 and/or at least a server 104 may generate the language processing model by any suitable method, including without limitation a natural language processing classification algorithm; language processing model may include a natural language process classification model that enumerates and/or derives statistical relationships between input term and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein, are statistical models with inference algorithms that that may be applied to the models. In such nodels, a hidden state to be estimated may include an association between an extracted word category of physiological data, a given relationship of such categories to compatible substance label 120, and/or a given category of compatible substance label 120. There may be a finite number of category of physiological data, a given relationship of such categories to compatible substance label 120, and/or a given category of compatible substance label 120 to which an extracted word may pertain; an HMM inference algorithm, such as the forward-backward algorithm or the Viterbi algorithm, may be used to estimate the most likely discrete state given a word or sequence of words. Language processing module 128 may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 1, language processing module 128 may use a corpus of documents to generate associations between language elements in a language processing module 128 and at least a server 104 may then use such associations to analyze words extracted from one or more documents and determine that the one or more documents indicate significance of a category of classified biomarker data, a given relationship of such categories to compatible substance label 120, and/or a given category of compatible substance label 120. In an embodiment, at least a server 104 may perform this analysis using a selected set of significant documents, such as documents identified by one or more experts as representing good science, good clinical analysis, or the like; experts may identify or enter such documents via graphical user interface 124, or may communicate identities of significant documents according to any other suitable method of electronic communication, or by providing such identity to other persons who may enter such identifications into at least a server 104. Documents may be entered into at least a server 104 by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, at least a server 104 may automatically obtain the document using such an identifier, for instance by submitting a request to a database or compendium of documents such as JSTOR as provided by Ithaka Harbors, Inc. of New York.

Continuing to refer to FIG. 1, whether an entry indicating significance of a category of classified biomarker data, a given relationship of such categories to compatible substance label 120, and/or a given category of compatible substance label 120 is entered via graphical user interface 124, alternative submission means, and/or extracted from a document or body of documents as described above, an entry or entries may be aggregated to indicate an overall degree of significance. For instance, each category of classified biomarker data, relationship of such categories to compatible substance label 120, and/or category of compatible substance label 120 may be given an overall significance score; overall significance score may, for instance, be incremented each time an expert submission and/or paper indicates significance as described above. Persons skilled in the art, upon reviewing the entirety of this disclosure will be aware of other ways in which scores may be generated using a plurality of entries, including averaging, weighted averaging, normalization, and the like. Significance scores may be ranked; that is, all categories of classified biomarker data, relationships of such categories to compatible substance label 120, and/or categories of compatible substance label 120 may be ranked according significance scores, for instance by ranking categories of classified biomarker data, relationships of such categories to compatible substance label 120, and/or categories of compatible substance label 120 higher according to higher significance scores and lower according to lower significance scores. Categories of classified biomarker data, relationships of such categories to compatible substance label 120, and/or categories of compatible substance label 120 may be eliminated from current use if they fail a threshold comparison, which may include a comparison of significance score to a threshold number, a requirement that significance score belong to a given portion of ranking such as a threshold percentile, quartile, or number of top-ranked scores. Significance scores may be used to filter outputs as described in further detail below; for instance, where a number of outputs are generated and automated selection of a smaller number of outputs is desired, outputs corresponding to higher significance scores may be identified as more probable and/or selected for presentation while other outputs corresponding to lower significance scores may be eliminated. Alternatively or additionally, significance scores may be calculated per sample type; for instance, entries by experts, documents, and/or descriptions of purposes of a given type of biomarker data or sample collection as described above may indicate that for that type of biomarker data or sample collection a first category of classified biomarker data, relationship of such category to compatible substance label 120, and/or category of compatible substance label 120 is significant with regard to that test, while a second category of classified biomarker data, relationship of such category to compatible substance label 120, and/or category of compatible substance label 120 is not significant; such indications may be used to perform a significance score for each category of classified biomarker data, relationship of such category to compatible substance label 120, and/or category of compatible substance label 120 is or is not significant per type of classified biomarker sample, which then may be subjected to ranking, comparison to thresholds and/or elimination as described above.

Still referring to FIG. 1, at least a server 104 may detect further significant categories of classified biomarker data, relationships of such categories to compatible substance label 120, and/or categories of compatible substance label 120 using machine-learning processes, including without limitation unsupervised machine-learning processes as described in further detail below; such newly identified categories, as well as categories entered by experts in free-form fields as described above, may be added to pre-populated lists of categories, lists used to identify language elements for language learning module, and/or lists used to identify and/or score categories detected in documents, as described above.

Continuing to refer to FIG. 1, in an embodiment, at least a server 104 may be configured, for instance as part of receiving the first training set 112, to associate at least a correlated first compatible substance label 120 with at least a category from a list of significant categories of compatible substance label 120. Significant categories of compatible substance label 120 may be acquired, determined, and/or ranked as described above. As a non-limiting example, compatible substance label 120 may be organized according to relevance to and/or association with a list of significant conditions. A list of significant conditions may include, without limitation, conditions having generally acknowledged impact on longevity and/or quality of life; this may be determined, as a non-limiting example, by a product of relative frequency of a condition within the population with years of life and/or years of able-bodied existence lost, on average, as a result of the condition. A list of conditions may be modified for a given person to reflect a family history of the person; for instance, a person with a significant family history of a particular condition or set of conditions, or a genetic profile having a similarly significant association therewith, may have a higher probability of developing such conditions than a typical person from the general population, and as a result at least a server 104 may modify list of significant categories to reflect this difference.

With continued reference to FIG. 1, at least a server 104 may be designed and configured to receive a second training set 132 including a plurality of second data entries. Each second data entry of the second training set 132 including at least a second element of second classified biomarker data 136 and at least a correlated compatible substance label 120. Second classified biomarker data 136 as used herein, may include any biomarker data that has been classified to a second dimension of the human body that may be separate from a first dimension of the human body. Dimension of the human body may include any of the dimensions of the human body as described above, including epigenetics, gut wall, microbiome, nutrients, genetics, and metabolism. For example and without limitation, where first classified biomarker data 116 may be classified to epigenetics, second classified biomarker data 136 may be classified to gut wall. In yet another non-limiting example, where first classified biomarker data 116 may be classified to microbiome, second classified biomarker data 136 may be classified to genetics. Correlation may include any correlation suitable for correlation of a first element of first classified biomarker data 116 and at least a correlated compatible substance label 120. Each second data entry of the second training set 132 includes at least a compatible substance label 120; at least a compatible substance label 120 may include any label suitable for use as compatible substance label 120 as described above.

With continued reference to FIG. 1, at least a server 104 may be configured, for instance as part of receiving second training set 132, to associate a compatible substance label 120 with at least a category from a list of significant categories of compatible substance label 120. This may be performed as described above for use of lists of significant categories with regard to first training set 112. Significance may be determined, and/or association with at least a category, may be performed for first training set 112 according to a first process as described above and for second training set 132 according to a second process.

With continued reference to FIG. 1, at least a server 104 may be configured, for instance as part of receiving second training set 132, to associate at least a correlated compatible substance label 120 with at least a category from a list of significant categories of compatible substance category labels. This may be done using expert input and utilizing any of the methodology as described above in reference to first training set 112.

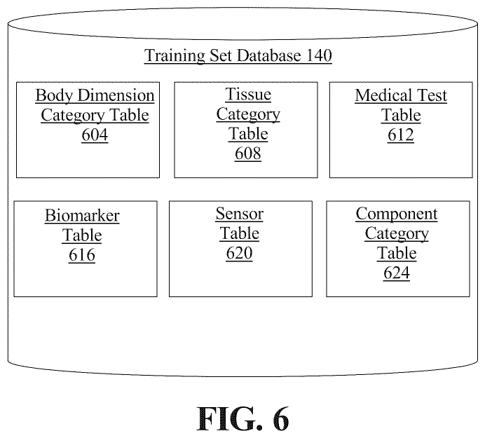

With continued reference to FIG. 1, at least a server 104 may be configured to receive component elements of training sets and utilize components to generate machine-learning models to select at least a compatible substance. Components may include individual training sets relating each of the six different body dimensions to correlated compatible substance label 120. For example and without limitation, at least a server 104 may be configured to receive a third training set including a plurality of third data entries, each third data entry of the plurality of third data entries including at least a third element of third classified biomarker data and at least a correlated compatible substance label 120. For example and without limitation, at least a server 104 may be configured to receive a fourth training set including a plurality of fourth data entries, each fourth data entry of the plurality of fourth data entries including at least a fourth element of fourth classified biomarker data and at least a correlated compatible substance label 120. For example and without limitation, at least a server 104 may be configured to receive a fifth training set including a plurality of fifth data entries, each fifth data entry of the plurality of fifth data entries including at least a fifth element of fifth classified biomarker data and at least a correlated compatible substance label 120. For example and without limitation, at least a server 104 may be configured to receive a sixth training set including a plurality of sixth data entries, each sixth data entry of the plurality of sixth data entries including at least a sixth element of sixth classified biomarker data and at least a correlated compatible substance label 120. At least a server 104 may receive training sets from training set database 140, as described below in more detail in reference to FIG. 6.

With continued reference to FIG. 1, at least a server 104 may be configured to receive component elements of training sets to generate machine-learning models to select at least a compatible substance label 120. Component elements of training sets may include training sets containing sub-sets of dimensional biomarker data related to correlated compatible substance label 120. For instance and without limitation, a first training set 112 including a first element of first classified biomarker data 116 relating to microbiome dimension may contain component elements with component elements containing sub-sets within dimension. In such an instance, microbiome dimension may be composed of component training sets that establish relations between sub-categories of microbiome biomarker data and compatible substance label 120. For instance and without limitation, microbiome dimension may include component training sets such as bacterial strains and compatible substance label 120, archaea strains and compatible substance label 120, fungi strains and compatible substance label 120, lactose breath tests and compatible substance label 120, methane breath tests and compatible substance label 120, fructose breath tests and compatible labels, stool cultures and compatible substance label 120 and the like.

With continued reference to FIG. 1, at least a server may be configured to select at least a first machine-learning model 148 as a function of the first training set 112 and the at least a biomarker datum. A machine learning process is a process that automatedly uses a body of data known as “training data” and/or a “training set” to generate an algorithm that will be performed by a computing device/module to produce outputs given data provided as inputs; this is in contrast to a non-machine learning software program where the commands to be executed are determined in advance by a user and written in a programming language.

With continued reference to FIG. 1, at least a server 104 may include at least a label learner 144, the at least a label learner 144 designed and configured to select at least a first machine-learning model 148 as a function of the first training set 112 and the at least a biomarker datum. At least a label learner 144 may include any hardware and/or software module. At least a label learner 144 is designed and configured to generate at least a compatible substance instruction set using the at least a biomarker datum, the first training set 112, and the at least a first machine-learning algorithm. A machine learning process is a process that automatedly uses a body of data known as “training data” and/or a “training set” to generate an algorithm that will be performed by a computing device/module to produce outputs given data provided as inputs; this is in contrast to a non-machine learning software program where the commands to be executed are determined in advance by a user and written in a programming language.

With continued reference to FIG. 1, at least a server 104 and/or at least a label learner 144 may be designed and configured to generate at least a first machine learning module using the first training set wherein the first machine-learning model outputs at least a compatible substance containing at least a compatible substance index value as a function of relating the at least a user biomarker datum to at least a compatible substance using the first training set and the at least a first machine-learning model. At least a first machine-learning model 148 may include one or more models that determine a mathematical relationship between biomarker data and compatible substance label 120. Such models may include without limitation model developed using linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

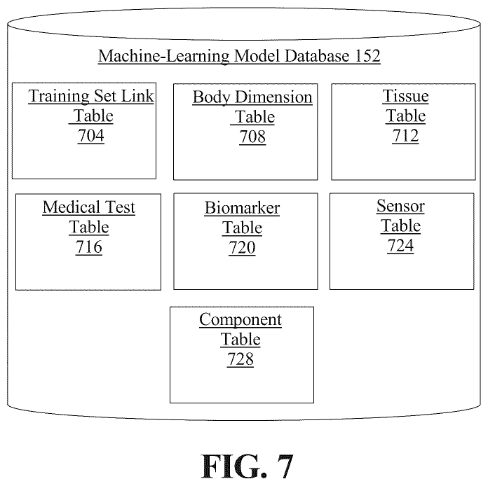

With continued reference to FIG. 1, at least a learner may select at least a first machine-learning model 148 from a machine-learning model database 152. This may be done as described below in more detail in reference to FIG. 7. First machine-learning model 148 may be selected as a function of the at least a first element of first classified biomarker datum contained within the first training set as described below in more detail in reference to FIG. 7.

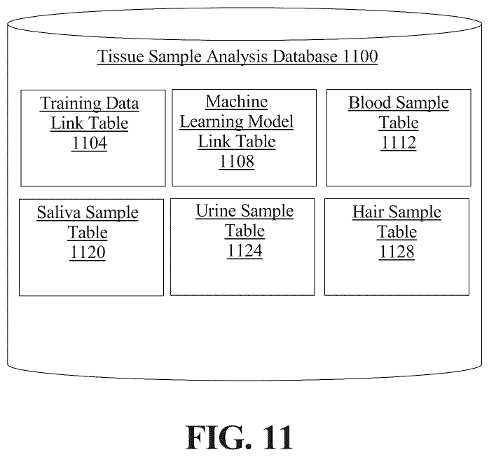

With continued reference to FIG. 1, at least a server 104 is configured to generate at least a compatible substance instruction set containing at least a compatible substance ranked as a function of the at least a compatible substance index value. Compatible substance instruction set may include a list of compatible substances, each containing a score indicating a particular percentage and/or indication of compatibility with a particular user. For example, a compatible substance may include a ranking that may include categories of compatible substances as described below in more detail in reference to FIG. 11.

With continued reference to FIG. 1, machine-learning algorithms may generate compatible substance instruction sets as a function of a classification of at least a compatible substance. Classification as used herein includes pairing or grouping of compatible substance label 120 as a function of a shared commonality. Classification may include for example, groupings, pairings, and/or trends between biomarker data and current compatible substance label 120, future compatible substance label 120, and the like. Machine-learning algorithms may include any and all algorithms as performed by any modules, described herein for at least a label learner 144. For example, machine-learning algorithms may relate fasting blood glucose readings of a user to user's future propensity to need to eliminate high starch foods. Machine-learning algorithms may examine precursor condition and future propensity to eliminate or necessitate consumption of a particular compatible element. For example, machine-learning algorithms may examine a user with a gene that codes for lactase insufficiency and future propensity to not be able to consume hard cheeses with low lactase quantities. Machine-learning algorithms may examine related food intolerances such as users who lack Bacteroides species and inability to digest dairy products as well as users who lack Lactobacillus species and inability to digest dairy products. Machine-learning algorithms may examine development of subsequent food recommendations. For example, machine-learning algorithms may examine low salivary progesterone and addition of sweet potatoes and subsequent addition of pumpkin after subsequent salivary tests reveal continued low progesterone levels.

Continuing to refer to FIG. 1, machine-learning algorithm used to generate first machine-learning model 148 may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors' algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 1, at least a label learner 144 may generate compatible substance label 120 using alternatively or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using any training set as described herein; the trained network may then be used to apply detected relationships between elements of classified biomarker data and compatible substance label 120.

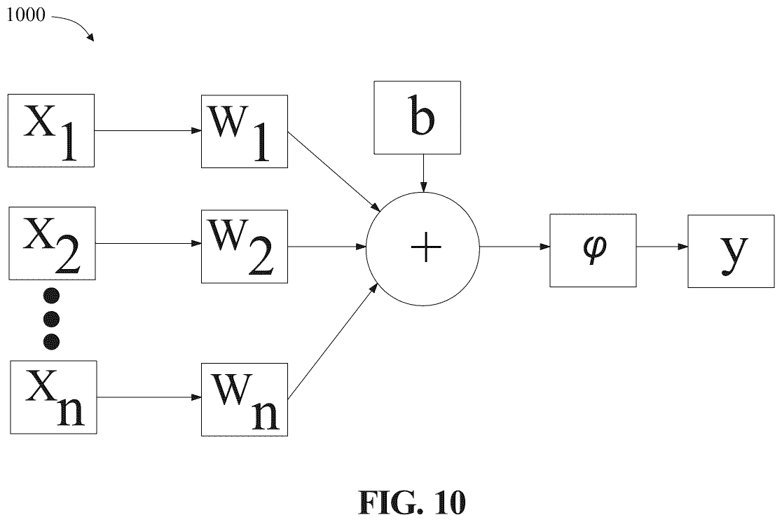

With continued reference to FIG. 1, generating at least a compatible substance instruction set may include retrieving at least a compatible substance index value from compatible substance index value database 156 and generating at least a compatible substance instruction set as a function of the at least a compatible substance index value. Compatible substance index value as used herein, is a value assigned to a compatible substance indicating a degree of compatibility between a first compatible element and a second compatible element for a user with any given biomarker datum. In an embodiment, compatible substance index value may be stored in a database or datastore as described below in more detail in reference to FIG. 10. In an embodiment, compatible substance index value scores may be calculated based on correlations between compatible substance category, compatible substance biochemistry, and impact of compatible substance on any given biomarker. In an embodiment, compatible substance index value may be ranked whereby a high compatible substance index value between any two compatible substances may indicate that for any two compatible substances a large percentage of individuals with a particular biomarker who tolerated a first compatible substance were able to then tolerate a second compatible substance. A low compatible substance index value between any two compatible elements may indicate that for any two compatible elements a small percentage of individuals with a particular biomarker who tolerated a first compatible substance were unable to then tolerate a second compatible substance. In an embodiment, a compatible substance index value may be evaluated as a function as a function of at least a biomarker datum from a user. For example, a compatible substance index value may contain a high index value for a first biomarker and a low index value for a second biomarker. Generating at least a compatible substance instruction set may include selecting a first compatible substance as a function of a first compatible substance index value and selecting at least a second compatible substance as a function of the first compatible substance index value and the second compatible substance index value.

Still referring to FIG. 1, in some embodiments, system 100 may include a camera. System 100 may, using a camera, capture an image, such as an image of a subject. Image may include a digital image. As used herein, a “camera” is a set of one or more devices configured to detect electromagnetic radiation. A camera may detect, in non-limiting examples, visible light, infrared light, and ultraviolet light. A camera may generate a representation of detected electromagnetic radiation, such as an image. In some cases, a camera may include one or more optics. Non-limiting examples of optics include spherical lenses, aspherical lenses, reflectors, polarizers, filters, windows, aperture stops, and the like. In some cases, a camera may include an image sensor. Exemplary non-limiting image sensors include digital image sensors, such as without limitation charge-coupled device (CCD) sensors and complimentary metal-oxide-semiconductor (CMOS) sensors, chemical image sensors, and analog image sensors, such as without limitation film. In some cases, a camera may be sensitive within a non-visible range of electromagnetic radiation, such as without limitation infrared. As used in this disclosure, “image data” is information representing at least a physical scene, space, and/or object. In some cases, image data may be generated by a camera. “Image data” may be used interchangeably through this disclosure with “image,” where image is used as a noun. An image may be optical, such as without limitation where at least an optic is used to generate an image of an object. An image may be material, such as without limitation when film is used to capture an image. An image may be digital, such as without limitation when represented as a bitmap. Alternatively, an image may be comprised of any media capable of representing a physical scene, space, and/or object. Alternatively, where “image” is used as a verb, in this disclosure, it refers to generation and/or formation of an image. In some embodiments, a camera may be configured to capture video. In some embodiments, a camera may be integrated into and/or connected to a computing device. For example, a camera may be integrated into and/or connected to a computing device which performs one or more steps described herein. In some embodiments, a camera may be a component of a remote device, such as a smartphone or other digital camera which is distinct from a computing device used to perform one or more steps described herein. In some embodiments, a computing device may, using a camera, capture an image. In some embodiments, capturing an image using a camera may include receiving the image from a remote device having a camera. In some embodiments, capturing an image using a camera may include receiving the image from a memory of a camera.

Still referring to FIG. 1, in some embodiments, system 100 may include an interface. An interface may be a component of a user device. A user device may include, in non-limiting examples, a smartphone, smartwatch, laptop computer, desktop computer, virtual reality device, or tablet. An interface may include an input interface and/or an output interface. An input interface may include one or more mechanisms for a computing device to receive data from a user such as, in non-limiting examples, a mouse, keyboard, button, scroll wheel, camera, microphone, switch, lever, touchscreen, trackpad, joystick, and controller. An output interface may include one or more mechanisms for a computing device to output data to a user such as, in non-limiting examples, a screen, speaker, and haptic feedback system. An output interface may be used to display one or more elements of data described herein. As used herein, a device “displays” a datum if the device outputs the datum in a format suitable for communication to a user. For example, a device may display a datum by outputting text or an image on a screen or outputting a sound using a speaker.

Still referring to FIG. 1, in some embodiments, system 100 may generate a body measurement. As used herein, a “body measurement” is a datum describing a body composition, a body mass, a body weight, a body dimension, a combination thereof, or a distribution thereof. As used herein, a “body dimension” is a datum describing a distance, area, or volume of a body or a section of a body. In a non-limiting example, a body dimension may include a height of a body. In another non-limiting example, a body dimension may include a circumference of a body's waist. In another non-limiting example, a body dimension may include a volume of a body. As used herein, a “body composition” is a datum describing the makeup of a body or a section of a body, or a distribution thereof. In a non-limiting example, a body composition may include a percent of a body which is made up of fat. In another non-limiting example, a body composition may include a percent of body fat of a body which is located in the body's legs. As used herein, “body mass” and “body mass metric” are used interchangeably to mean a datum describing the mass of a body, a section of a body or a subset of tissues of a body, or a distribution of the mass of a body a section of a body or a subset of tissues of a body. In a non-limiting example, a body mass metric may include a measurement of a lean body mass. In another non-limiting example, a body mass metric may include a measurement of the weight of bones of a body. As used herein, a “body weight” is a datum describing the weight of a body or a section of a body, or a distribution thereof. In some embodiments, body measurement includes a metric selected from the list consisting of visceral fat content, height, weight, body mass index, lean body mass, body water percentage, and bone density.

Still referring to FIG. 1, system 100 may determine body measurement using a body measurement machine learning model. A body measurement machine learning model may be trained using a supervised learning algorithm. A body measurement machine learning model may include a neural network, such as a convolutional neural network and/or a deep neural network. A body measurement machine learning model may be trained on a training dataset including example images, associated with example body measurements. Such a training dataset may be obtained by, for example, collecting and associating images of bodies with measurements of such bodies. In a non-limiting example, a subject may wear a suit which measures various aspects of the subject's body such as dimensions and composition of the subject's body, and images may be captured of such subject. Such measurements and images may be collected for a plurality of subjects and used to assemble a training dataset. Once body measurement machine learning model is trained, it may be used to determine a body measurement. System 100 may input an image into body measurement machine learning model, and system 100 may receive a body measurement from the model.

Still referring to FIG. 1, in some embodiments, system 100 may use a machine vision system to generate a body measurement. In some embodiments, a machine vision system may include at least a camera. A machine vision system may use images, such as images from at least a camera, to make a determination about a scene, space, and/or object. For example, in some cases a machine vision system may be used for world modeling or registration of objects within a space. In some cases, registration may include image processing, such as without limitation object recognition, feature detection, edge/corner detection, and the like. Non-limiting example of feature detection may include scale invariant feature transform (SIFT), Canny edge detection, Shi Tomasi corner detection, and the like. In some cases, registration may include one or more transformations to orient a camera frame (or an image or video stream) relative a three-dimensional coordinate system; exemplary transformations include without limitation homography transforms and affine transforms. In an embodiment, registration of first frame to a coordinate system may be verified and/or corrected using object identification and/or computer vision, as described above. For instance, and without limitation, an initial registration to two dimensions, represented for instance as registration to the x and y coordinates, may be performed using a two-dimensional projection of points in three dimensions onto a first frame, however. A third dimension of registration, representing depth and/or a z axis, may be detected by comparison of two frames; for instance, where first frame includes a pair of frames captured using a pair of cameras (e.g., stereoscopic camera also referred to in this disclosure as stereo-camera), image recognition and/or edge detection software may be used to detect a pair of stereoscopic views of images of an object; two stereoscopic views may be compared to derive z-axis values of points on object permitting, for instance, derivation of further z-axis points within and/or around the object using interpolation. This may be repeated with multiple objects in field of view, including without limitation environmental features of interest identified by object classifier and/or indicated by an operator. In an embodiment, x and y axes may be chosen to span a plane common to two cameras used for stereoscopic image capturing and/or an xy plane of a first frame; a result, x and y translational components and ϕ may be pre-populated in translational and rotational matrices, for affine transformation of coordinates of object, also as described above. Initial x and y coordinates and/or guesses at transformational matrices may alternatively or additionally be performed between first frame and second frame, as described above. For each point of a plurality of points on object and/or edge and/or edges of object as described above, x and y coordinates of a first stereoscopic frame may be populated, with an initial estimate of z coordinates based, for instance, on assumptions about object, such as an assumption that ground is substantially parallel to an xy plane as selected above. Z coordinates, and/or x, y, and z coordinates, registered using image capturing and/or object identification processes as described above may then be compared to coordinates predicted using initial guess at transformation matrices; an error function may be computed using by comparing the two sets of points, and new x, y, and/or z coordinates, may be iteratively estimated and compared until the error function drops below a threshold level. In some cases, a machine vision system may use a classifier, such as any classifier described throughout this disclosure.

Still referring to FIG. 1, an exemplary machine vision camera is an OpenMV Cam H7 from OpenMV, LLC of Atlanta, Georgia, U.S.A. OpenMV Cam comprises a small, low power, microcontroller which allows execution of machine vision applications. OpenMV Cam comprises an ARM Cortex M7 processor and a 640×480 image sensor operating at a frame rate up to 150 fps. OpenMV Cam may be programmed with Python using a Remote Python/Procedure Call (RPC) library. OpenMV CAM may be used to operate image classification and segmentation models, such as without limitation by way of TensorFlow Lite; detection motion, for example by way of frame differencing algorithms; marker detection, for example blob detection; object detection, for example face detection; eye tracking; person detection, for example by way of a trained machine learning model; camera motion detection, for example by way of optical flow detection; code (barcode) detection and decoding; image capture; and video recording.

Still referring to FIG. 1, system 100 may process an image and/or a plurality of images using an image processing module. Image processing may be performed, for example, in order to make images suitable for input into body measurement machine learning model. As used in this disclosure, an “image processing module” is a component of a device designed to process digital images. In an embodiment, image processing module may include a plurality of software algorithms that can analyze, manipulate, or otherwise enhance plurality of images, such as, without limitation, a plurality of image processing techniques as described below. In another embodiment, image processing module may slow include hardware components such as, without limitation, one or more graphics processing units (GPUs) that can accelerate the processing of large amount of images. In some cases, image processing module may be implemented with one or more image processing libraries such as, without limitation, OpenCV, PIL/Pillow, ImageMagick, and the like.

Still referring to FIG. 1, image processing module is configured to receive plurality of images from at least an camera. In a non-limiting example, image processing module may be configured to receive the plurality of images by generating a first image capture parameter, transmitting a command to at least an camera to take at least a first image of the plurality of images with the first image capture parameter, generating a second image capture parameter, transmitting a command to at least an camera to take at least a second image of the plurality of images with the second image capture parameter, and receiving, from at least an camera, at least a first image and at least second image. In another non-limiting example, plurality of images may be taken by at least an camera using the same image capture parameter. Image capture parameter may be generated as a function of user input.

Still referring to FIG. 1, at least an image may be transmitted from at least an camera to image processing module via any suitable electronic communication protocol, including without limitation packet-based protocols such as transfer control protocol-internet protocol (TCP-IP), file transfer protocol (FTP) or the like. In case of user device of image capturing device, plurality of images may be transmitted via a text messaging service such as simple message service (SMS) or the like. plurality of images may be received via a portable memory device such as a disc or “flash” drive, via local and/or near-field communication (NFC), or according to any other direct or indirect means for transmission and/or transfer of digital images. Receiving plurality of images may include retrieval of plurality of images from a data store containing plurality of images as described below; for instance, and without limitation, plurality of images may be retrieved using a query that, for instance, specifies a timestamp that one or more images may be required to match.

Still referring to FIG. 1, image processing module is configured to process the plurality of images. In an embodiment, image processing module may be configured to compress and/or encode plurality of images to reduce the file size and storage requirements while maintaining the essential visual information (e.g., visual information of a body) need for further processing steps as described below. In an embodiment, compression and/or encoding of plurality of images may facilitate faster transmission of plurality of images. In some cases, image processing module may be configured to perform a lossless compression on plurality of images, wherein the lossless compression may maintain the original image quality of plurality of images. In a non-limiting example, image processing module may utilize one or more lossless compression algorithms, such as, without limitation, Huffman coding, Lempel-Ziv-Welch (LZW), Run-Length Encoding (RLE), and/or the like to identify and remove redundancy in each image of plurality of images without losing any information. In such embodiment, compressing and/or encoding each image of plurality of images may include converting the file format of each image into PNG, GIF, lossless JPEG2000 or the like. In an embodiment, plurality of images compressed via lossless compression may be perfectly reconstructed to the original form (e.g., original image resolution, dimension, color representation, format, and the like) of plurality of images. In other cases, image processing module may be configured to perform a lossy compression on plurality of images, wherein the lossy compression may sacrifice some image quality of plurality of images to achieve higher compression ratios. In a non-limiting example, image processing module may utilize one or more lossy compression algorithms, such as, without limitation, Discrete Cosine Transform (DCT) in JPEG or Wavelet Transform in JPEG2000, discard some less significant information within plurality of images, resulting in a smaller file size but a slight loss of image quality of plurality of images. In such embodiment, compressing and/or encoding each image of plurality of images may include converting the file format of each image into JPEG, WebP, lossy JPEG2000, or the like.

Still referring to FIG. 1, in an embodiment, processing plurality of images may include determining a degree of quality of depiction of a body for each image of plurality of images. As used in this disclosure, a “degree of quality of depiction” of a body is the degree to which image clearly depicts a body. In an embodiment, image processing module may determine a degree of blurriness of each image of plurality of images. In a non-limiting example, image processing module may perform a blur detection by taking a Fourier transform, or an approximation such as a Fast Fourier Transform (FFT) of each image of plurality of images and analyzing a distribution of low and high frequencies in the resulting frequency-domain depiction of each image of plurality of images; for instance, and without limitation, numbers of high-frequency values below a threshold level may indicate blurriness. In another non-limiting example, detection of blurriness may be performed by convolving each image of plurality of images, a channel of each image of plurality of images, or the like with a Laplacian kernel; for instance, and without limitation, this may generate a numerical score reflecting a number of rapid changes in intensity shown in each image, such that a high score indicates clarity and a low score indicates blurriness. In some cases, blurriness detection may be performed using a Gradient-based operator, which measures operators based on the gradient or first derivative of each image of plurality of images, based on the hypothesis that rapid changes indicate sharp edges in the image, and thus are indicative of a lower degree of blurriness. In some cases, blur detection may be performed using Wavelet-based operator, which takes advantage of the capability of coefficients of the discrete wavelet transform to describe the frequency and spatial content of plurality of images. In some cases, blur detection may be performed using statistics-based operators take advantage of several image statistics as texture descriptors in order to compute a focus level. In other cases, blur detection may be performed by using discrete cosine transform (DCT) coefficients in order to compute a focus level of each image of plurality of images from its frequency content. Additionally, or alternatively, image processing module may be configured to rank plurality of images according to degree of quality of depiction of a body and select a highest-ranking image from plurality of images.

Still referring to FIG. 1, processing plurality of images may include enhancing at least an image containing a body via a plurality of image processing techniques to improve the quality (or degree of quality of depiction) of at least an image for better processing and analysis as described further in this disclosure. In an embodiment, image processing module may be configured to perform a noise reduction operation on at least an image containing a bod, wherein the noise reduction operation may remove or minimize noise (arises from various sources, such as sensor limitations, poor lighting conditions, image compression, and/or the like), resulting in a cleaner and more visually coherent image. In some cases, noise reduction operation may be performed using one or more image filters; for instance, and without limitation, noise reduction operation may include Gaussian filtering, median filtering, bilateral filtering, and/or the like. Noise reduction operation may be done, by image processing module, by averaging or filtering out pixel values in neighborhood of each pixel of at least an image to reduce random variations.

Still referring to FIG. 1, in another embodiment, image processing module may be configured to perform a contrast enhancement operation on at least an image containing a body. In some cases, at least an image may exhibit low contrast, making a body difficult to distinguish from the background. Contrast enhancement operation may improve the contrast of at least an image containing a body by stretching the intensity range of at least an image and/or redistributing the intensity values (i.e., degree of brightness or darkness of a pixel in at least an image). In a non-limiting example, intensity value may represent the gray level or color of each pixel, scale from 0 to 255 in intensity range for an 8-bit image, and scale from 0 to 16,777,215 in a 24-bit color image. In some cases, contrast enhancement operation may include, without limitation, histogram equalization, adaptive histogram equalization (CLAHE), contrast stretching, and/or the like. image processing module may be configured to adjust the brightness and darkness levels within the at least an image to make a body more distinguishable (i.e., increase degree of quality of depiction). Additionally, or alternatively, image processing module may be configured to perform a brightness normalization operation to correct variations in lighting conditions (i.e., uneven brightness levels). In some cases, at least an image may include a consistent brightness level across the entire a body after brightness normalization operation performed by image processing module. In a non-limiting example, image processing module may perform a global or local mean normalization, where the average intensity value of the entire image or a body may be calculated and used to adjust the brightness levels.

Still referring to FIG. 1, in some embodiments, image processing module may be configured to perform a color space conversion operation to increase degree of quality of depiction. In a non-limiting example, in case of color image (i.e., RGB image), image processing module may be configured to convert RGB image to grayscale or HSV color space. Such conversion may emphasize the differences in intensity values between a body and the background. image processing module may further be configured to perform an image sharpening operation such as, without limitation, unsharp masking, Laplacian sharpening, high-pass filtering, and/or the like. image processing module may use image sharpening operation to enhance the edges and fine details related to a body within at least an image by emphasizing high-frequency components within at least an image.

Still referring to FIG. 1, processing plurality of images may include isolating a body from at least an image as a function of plurality of image processing techniques. At least an image may include highest-ranking image selected by image processing module as described above. In an embodiment, plurality of image processing techniques may include one or more morphological operations, wherein the morphological operations are techniques developed based on set theory, lattice theory, topology, and random functions used for processing geometrical structures using a structuring element. A “structuring element,” for the purpose of this disclosure, is a small matrix or kernel that defines a shape and size of a morphological operation. In some cases, structing element may be centered at each pixel of at least an image and used to determine an output pixel value for that location. In a non-limiting example, isolating a body from at least an image may include applying a dilation operation, wherein the dilation operation is a basic morphological operation configured to expand or grow the boundaries of objects in at least an image. In another non-limiting example, isolating a body from at least an image may include applying an erosion operation, wherein the erosion operation is a basic morphological operation configured to shrink or erode the boundaries of objects in at least an image. In another non-limiting example, isolating a body from at least an image may include applying an opening operation, wherein the opening operation is a basic morphological operation configured to remove small objects or thin structures from at least an image while preserving larger structures. In a further non-limiting example, isolating a body from at least an image may include applying a closing operation, wherein the closing operation is a basic morphological operation configured to fill in small gaps or holes in objects in at least an image while preserving the overall shape and size of the objects. These morphological operations may be performed by image processing module to enhance the edges of objects, remove noise, or fill gaps in a body before further processing.

Still referring to FIG. 1, in an embodiment, isolating a body from at least an image may include utilizing an edge detection technique, which may detect one or more shapes defined by edges. An “edge detection technique,” as used in this disclosure, includes a mathematical method that identifies points in a digital image, such as, without limitation, at least an image, at which the image brightness changes sharply and/or has discontinuities. In an embodiment, such points may be organized into straight and/or curved line segments, which may be referred to as “edges.” Edge detection technique may be performed, by image processing module, using any suitable edge detection algorithm, including without limitation Canny edge detection, Sobel operator edge detection, Prewitt operator edge detection, Laplacian operator edge detection, and/or Differential edge detection. Edge detection technique may include phase congruency-based edge detection, which finds all locations of an image where all sinusoids in the frequency domain, for instance as generated using a Fourier decomposition, may have matching phases which may indicate a location of an edge. Edge detection technique may be used to detect a shape of a body; in an embodiment, edge detection technique may be used to find closed figures formed by edges.

Still referring to FIG. 1, in some embodiments, isolating a body from at least an image may include determining a region of interest (ROI) via edge detection technique. As used in this disclosure, a “region of interest” is a specific area within a digital image that contains information relevant to a body. In a non-limiting example, image information located outside ROI may include irrelevant or extraneous information such as, without limitation, a background. Such portion of image containing irrelevant or extraneous information may be disregarded by image processing module. In some cases, ROI may vary in size, shape, and/or location within at least an image. In a non-limiting example ROI may be presented as a rectangular bounding box (length×width) around a body on at least an image. In some cases, ROI may specify one or more coordinates of one or more corners of rectangular bounding box, and/or length and/or width of rectangular bounding box around a body on at least an image. image processing module may then be configured to isolate a body from the at least an image based on ROI. In a non-limiting example, and without limitation, image processing module may crop at least an image according to rectangular bounding box around a body.

Still referring to FIG. 1, image processing module may be configured to perform a connected component analysis (CCA) on at least an image for a body isolation. As used in this disclosure, a “connected component analysis (CCA),” also known as connected component labeling, is an image processing technique used to identify and label connected regions within a binary image (i.e., an image which each pixel having only two possible values: 0 or 1, black or white, or foreground and background). “Connected regions,” as described herein, is a group of adjacent pixels that share the same value and are connected based on a predefined neighborhood system such as, without limitation, 4-connected or 8-connected neighborhoods. In some cases, image processing module may convert at least an image into a binary image via a thresholding process, wherein the thresholding process may involve setting a threshold value that separates the pixels of at least an image corresponding to the body (foreground) from those corresponding to the background. Pixels with intensity values above the threshold may be set to 1 (white) and those below the threshold may be set to 0 (black). In an embodiment, CCA may be employed to detect and extract a body by identifying a plurality of connected regions that exhibit specific properties or characteristics of a body. image processing module may then filter plurality of connected regions by analyzing plurality of connected regions properties such as, without limitation, area, aspect ratio, height, width, perimeter, and/or the like. In a non-limiting example, connected components that closely resemble the dimensions and aspect ratio of a body may be retained, by image processing module, while other components may be discarded.

Still referring to FIG. 1, in an embodiment, isolating a body from at least an image may include segmenting a body into a plurality of a body sub-regions. Segmenting a body into plurality of a body sub-regions may include segmenting a body as a function of ROI and/or CCA via an image segmentation process. As used in this disclosure, an “image segmentation process” is a process for partition a digital image, such as, without limitation, an image, into one or more segments, wherein each segment represents a distinct part of the image. Image segmentation process may change the representation of plurality of images. Image segmentation process may be performed, by image processing module, via one or more image segmentation techniques. In a non-limiting example, image processing module may perform a region-based segmentation, wherein the region-based segmentation involves growing regions from one or more seed points or pixels on at least an image based on a similarity criterion. Similarity criterion may include, without limitation, color, intensity, texture, and/or the like. In a non-limiting example, region-based segmentation may include region growing, region merging, watershed algorithms, and the like.

Still referring to FIG. 1, in some embodiments, system 100 may determine a compatible substance as a function of a body measurement. As used herein, a substance is a “compatible substance” for a subject if ingestion of the substance by the subject improves the health of the subject. In some embodiments, determination of a compatible substance may include gathering dietary information of a subject, making a comparison between a body measurement of the subject and a typical body measurement of a healthy individual, and identifying a directional dietary change based on such comparison.

Still referring to FIG. 1, in some embodiments, system 100 may gather dietary information of a subject. dietary information may be received from a dietary information source. As used herein, a “dietary information source” is a device, entity, system, or combination thereof containing dietary information, configured to detect dietary information, configured to transmit dietary information, or a combination thereof. In some embodiments, dietary information source may include one or more user devices, databases, computing devices, and/or users. In non-limiting examples, user devices may include smartphones, smartwatches, tablets, and computers. In some embodiments, a dietary information source may include a physical or digital form such as a form on a website or in an application. Exemplary forms include forms asking a subject to input items the user ate recently. As another non-limiting example, a dietary information source may include a computing device configured to receive dietary information using digital tracking, such as gathering information using a device fingerprint that allows a user device to be tracked across the internet. As a non-limiting example, a device fingerprint may allow a user device to be tracked to a website such as a website which recommends recipes. In some embodiments, dietary information may be received from a third party. In a non-limiting example, a third party may operate a database including dietary information, system 100 may request dietary information from the database using an application programming interface (API), and system 100 may receive from the database, or a computing device associated with the database, dietary information.

Still referring to FIG. 1, dietary information may be input through an interface. An interface may include a graphical user interface (GUI). An interface may include a touch-screen GUI interface. An interface may include a computing device configured to receive an input from a user. In some embodiments. an interface may be configured to prompt a user for an input. In a non-limiting example, an interface may request that a user input information as to food items the user eats most frequently.

Still referring to FIG. 1, in some embodiments, determining a compatible substance may include comparing a body measurement of a subject to a typical body measurement of a healthy individual. Such a comparison may be made while controlling one or more other variables. In a non-limiting example, system 100 may compare a weight of a subject's body with weights of bodies of healthy individuals within a certain height range of the subject. In another non-limiting example, system 100 may compare a waist circumference of a subject's body with a waist circumference of bodies of healthy individuals within a certain height range of the subject.

Still referring to FIG. 1, in some embodiments, determining a compatible substance may include identifying a directional dietary change based on a comparison between body measurements. Such a dietary change may include, for example, swapping one food of a diet of a subject for another. For example, a high calorie food may be swapped for a low calorie food. In another example, a food with a high amount of added sugar may be swapped for a food with a low amount of added sugar. In some embodiments, a first food may be swapped to a second food which has one or more properties of the first food. For example, a first cooking oil may be swapped for a second cooking oil. In another example, a first bread may be swapped for a second bread. In another example, a first common snack food may be swapped for a second common snack food. For example, if a body measurement of a subject indicates that the subject is heavier than a typical healthy individual within a certain height range of the subject, then a high calorie food of the subject's diet may be swapped for a low calorie food. In another example, if a body measurement of a subject indicates that the subject is healthy, then in some embodiments, no alteration to subject's diet is made. In some embodiments, a compatible substance may include a substance of a post-swap diet of a subject.

Still referring to FIG. 1, in some embodiments, determination of a compatible substance may take into account subject input and/or subject feedback. For example, a first compatible substance may be determined and displayed to a user, such as a subject, the user may provide feedback, and a second compatible substance may be determined as a function of such feedback. For example, several compatible substances may be determined, and subject may select from such compatible substances. In some embodiments, a compatible substance may be displayed. Display of information is described below.

Still referring to FIG. 1, in some embodiments, system 100 may receive a subject health datum. As used herein, a “subject health datum” is a datum describing an aspect of health of a subject, a change in health of a subject, or both. A subject health datum may include data describing, in non-limiting examples, a sleep pattern of a subject, a dietary change of a subject, a water intake of a subject, a relationship of a subject, a mental status of a subject, a change in the amount of exercise a subject gets, an injury of a subject, and a medicine taken by the subject. In some embodiments, system 100 may determine a body measurement as a function of a health datum. For example, after a first compatible substance is determined based on a first image, system 100 may receive a subject health datum and, using a camera, capture a second image as a function of the subject health datum; generate a second body measurement as a function of the second image using the trained body measurement machine learning model; determine a second compatible substance as a function of the second body measurement; and, using a user interface, display the second compatible substance. In some embodiments, system 100 may periodically determine body measurements of a subject.

Still referring to FIG. 1, in some embodiments, system 100 may identify a body measurement impact ingredient. As used herein, a “body measurement impact ingredient” is a substance whose ingestion is associated with a change in a body measurement. In some embodiments, a body measurement impact ingredient may be determined as a function of a selection of a body measurement. In some embodiments, a body measurement impact ingredient may be determined as a function of a body measurement of a subject which is different than that of a typical healthy individual. A body measurement impact ingredient may be determined based on, for example, studies on health effects of various foods. In some embodiments, a body measurement impact ingredient may be determined based on health effects of ingredients of such food. In some embodiments, a body measurement impact ingredient may be determined based on nutrition information of such food, such as sugar content, fat content, protein content, and calorie count of such food. In some embodiments, system 100 may generate a nutrient plan as a function of a body measurement impact ingredient. As used herein, a “nutrient plan” is a data structure including data describing a diet and an amount of a nutrient provided by the diet. For example, a nutrient plan may indicate foods included in a diet, and nutrient contributions of such foods across a plurality of nutrients. In some embodiments, a nutrient plan and/or a body measurement impact ingredient may be displayed as described below.

Still referring to FIG. 1, in some embodiments, system 100 may generate a digital avatar as a function of a body measurement. As used herein, a “digital avatar” is a visual representation of a human displayed using a computing device. In some embodiments, a digital avatar may include a visual representation of a subject described by a body measurement. For example, a digital avatar may include features which visually resemble those of a subject. Such features may be generated using a machine vision system as described above. In some embodiments, a digital avatar may have a body measurement of a subject. Such body measurement may be made accounting for a difference in scale of a digital avatar and a subject. In a non-limiting example, a digital avatar may be generated such that a ratio of height of a subject to waist circumference of the subject is used to determine such ratio of the digital avatar. In some embodiments, a digital avatar may include a 2 dimensional digital avatar. In some embodiments, a digital avatar may include a 3 dimensional digital avatar. In some embodiments, a digital avatar may be animated.

Still referring to FIG. 1, in some embodiments, system 100 may receive a body measurement adjustment datum. In some embodiments, body measurement adjustment datum may be received from a user interface. As used herein, a “body measurement adjustment datum” is a datum which sets a value of a body measurement, adjusts a value of a body measurement, or both. For example, a body measurement adjustment datum may be input by a user in order to correct an error as to a calculated body measurement. In some embodiments, a digital avatar may be generated as a function of a body measurement adjustment datum. In a non-limiting example, a first digital avatar may be generated and displayed to a user, the user may input a body measurement adjustment datum using a user interface, and a second digital avatar may be generated and displayed to the user as a function of the body measurement adjustment datum.

Still referring to FIG. 1, in some embodiments, system 100 may generate a health improvement body measurement estimation. As used herein, a “health improvement body measurement estimation” is a datum describing an estimate of a body measurement of a subject after the subject performs an action. In a non-limiting example, a health improvement body measurement estimation may include an estimate that a muscle mass of a user will increase by 10% if the user consumes a particular amount of protein daily. In another non-limiting example, a health improvement body measurement estimation may include an estimate that a mass of a user will decrease to a particular level if the user jogs 2 miles daily for 6 months. In some embodiments, a health improvement body measurement estimation may include an absolute number. In some embodiments, a health improvement body measurement estimation may include a number relative to a body measurement. In some embodiments, a digital avatar may be generated as a function of a health improvement body measurement estimation and displayed to a user. For example, a first digital avatar indicating a current body measurement may be displayed alongside a second digital avatar indicating a body measurement which may be attainable for a subject.

Still referring to FIG. 1, in some embodiments, system 100 may determine a medical condition risk datum as a function of a body measurement. As used herein, a “medical risk datum” is a datum which indicates that a subject has a medical condition, is at risk of developing a medical condition, or both. For example, a medical risk datum may be determined if a subject's weight far exceeds that of a typical healthy individual of the subject's height. In some embodiments, system 100 may generate a medical risk datum as a function of a body measurement indicating a body fat distribution of a subject. A medical risk datum may be displayed as described below.

Still referring to FIG. 1, in some embodiments, system 100 may display one or more elements of data described herein. For example, system 100 may display a compatible substance. In some embodiments, display of an element of data may include generation of a user interface, such as a graphical user interface (GUI), and display of the element of data using the user interface. In some embodiments, a user interface may be displayed using an output interface of a user device, such as a device of a user which is remote to a computing device configured to perform one or more steps described herein. In some embodiments, a computing device of system 100 may transmit data to a user device configuring the user device to display an element of data such as a compatible substance. In a non-limiting example, system 100 may transmit to a user device HTML code configuring the user device to display a web page including a compatible substance. In some embodiments, data may be displayed in the form of one or more visual elements. A visual element data structure may include a visual element. As used herein, a “visual element” is a datum that is displayed visually to a user. In some embodiments, a visual element data structure may include a rule for displaying visual element. In some embodiments, a visual element data structure may be determined as a function of a body measurement and/or a compatible substance. In some embodiments, a visual element data structure may be determined as a function of an item from the list consisting of an image, a body measurement, a compatible substance, a subject health datum, a body measurement impact ingredient, a digital avatar, and a medical risk datum. In a non-limiting example, a visual element data structure may be generated such that visual element describing or highlighting a compatible substance is displayed to a user.

Still referring to FIG. 1, in some embodiments, visual element may include one or more elements of text, images, shapes, charts, particle effects, interactable features, and the like. For example, a visual element may include an interactable feature into which a user may input data used to generate a body measurement adjustment datum.

Still referring to FIG. 1, a visual element data structure may include rules governing if or when visual element is displayed. In a non-limiting example, a visual element data structure may include a rule causing a visual element describing body measurement to be displayed when a user selects body measurement using a graphical user interface (GUI).

Still referring to FIG. 1, a visual element data structure may include rules for presenting more than one visual element, or more than one visual element at a time. In an embodiment, about 1, 2, 3, 4, 5, 10, 20, or 50 visual elements are displayed simultaneously.

Still referring to FIG. 1, a visual element data structure rule may apply to a single visual element or datum, or to more than one visual element or datum. For example, a visual element data structure may rank visual elements and/or other data and/or apply numerical values to them, and a computing device may display a visual element as a function of such rankings and/or numerical values. A visual element data structure may apply rules based on a comparison between such a ranking or numerical value and a threshold.=

Still referring to FIG. 1, in some embodiments, visual element may be interacted with. For example, visual element may include an interface, such as a button or menu. In some embodiments, visual element may be interacted with using a user device such as a smartphone.

Still referring to FIG. 1, in some embodiments, system 100 may transmit visual element data structure to a user device. In some embodiments, visual element data structure may configure a user device to display visual element. In some embodiments, visual element data structure may cause an event handler to be triggered in an application of a user device such as a web browser. In some embodiments, triggering of an event handler may cause a change in an application of a user device such as display of visual element.

Still referring to FIG. 1, in some embodiments, system 100 may transmit visual element to a display. A display may communicate visual element to user. A display may include, for example, a smartphone screen, a computer screen, or a tablet screen. A display may be configured to provide a visual interface. A visual interface may include one or more virtual interactive elements such as, without limitation, buttons, menus, and the like. A display may include one or more physical interactive elements, such as buttons, a computer mouse, or a touchscreen, that allow user to input data into the display. Interactive elements may be configured to enable interaction between a user and a computing device. In some embodiments, a visual element data structure is determined as a function of data input by user into a display.

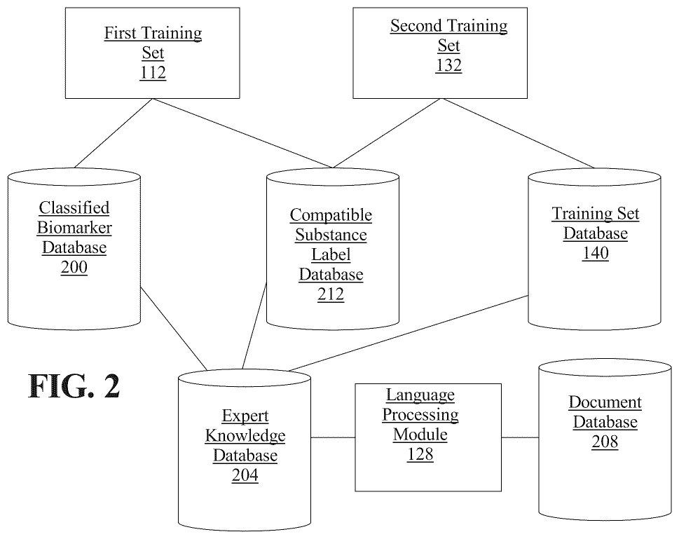

Referring now to FIG. 2, data incorporated in first training set 112 and/or second training set 132 may be incorporated in one or more databases. As a non-limiting example, one or elements of classified biomarker data may be stored in and/or retrieved from a classified biomarker database 200. A classified biomarker database 200 may include any data structure for ordered storage and retrieval of data, which may be implemented as a hardware or software module. A classified biomarker database 200 may be implemented, without limitation, as a relational database, a key-value retrieval datastore such as a NOSQL database, or any other format or structure for use as a datastore that a person skilled in the art would recognize as suitable upon review of the entirety of this disclosure. A classified database 200 may include a plurality of data entries and/or records corresponding to elements of biomarker data as described above. Data entries and/or records may describe, without limitation, data concerning particular biological samples that have been collected; entries may describe reasons for collection of samples, such as without limitation one or more conditions being tested for, which may be listed with related body dimensions. Data entries may include compatible substance label 120 and/or other descriptive entries describing results of evaluation of past physiological data, including results of evaluation by experts including any of the experts as described herein. Such conclusions may have been generated by system 100 in previous iterations of methods, with or without validation of correctness by medical professionals. Data entries in classified biomarker database 200 may be flagged with or linked to one or more additional elements of information, which may be reflected in data entry cells and/or in linked tables such as tables related by one or more indices in a relational database; one or more additional elements of information may include data associating a biomarker sample and/or a person from whom a biomarker sample was extracted or received with one or more cohorts, including demographic groupings such as ethnicity, sex, age, income, geographical region, or the like, one or more common traits or physiological attributes shared with other persons having physiological samples reflected in other data entries, or the like. Additional elements of information may include one or more classified categories of biomarker data as described above. Additional elements of information may include descriptions of particular methods used to obtain physiological samples, such as without limitation physical extraction of blood samples or the like, capture of data with one or more sensors, and/or any other information concerning provenance and/or history of data acquisition. Persons skilled in the art, upon reviewing the entirety of this disclosure, will be aware of various ways in which data entries in a classified biomarker database 200 may reflect categories, cohorts, and/or populations of data consistently with this disclosure.

With continued reference to FIG. 2, at least a server 104 and/or another device in system 100 may populate one or more fields in classified biomarker database 200 using expert information, which may be extracted or retrieved from an expert knowledge database 204. An expert knowledge database 204 may include any data structure and/or data store suitable for use as a biomarker database 200 as described above. Expert knowledge database 204 may include data entries reflecting one or more expert submissions of data such as may have been submitted according to any process described above in reference to FIG. 1, including without limitation by using graphical user interface 124. Expert knowledge database may include one or more fields generated by language processing module 128, such as without limitation fields extracted from one or more documents as described above. For instance, and without limitation, one or more categories of biomarker data and/or related compatible substance label 120 and/or categories of compatible substance label 120 associated with an element of classified biomarker data as described above may be stored in generalized from in an expert knowledge database 204 and linked to, entered in, or associated with entries in a biomarker database 200. Documents may be stored and/or retrieved by at least a server 104 and/or language processing module 128 in and/or from a document database 208; document database 208 may include any data structure and/or data store suitable for use as biomarker database 200 as described above. Documents in document database 208 may be linked to and/or retrieved using document identifiers such as URI and/or URL data, citation data, or the like; persons skilled in the art, upon reviewing the entirety of this disclosure, will be aware of various ways in which documents may be indexed and retrieved according to citation, subject matter, author, date, or the like as consistent with this disclosure.

With continued reference to FIG. 2, a compatible substance label 120 database 212, which may be implemented in any manner suitable for implementation of classified biomarker database 200, may be used to store compatible substance label 120 used in system 100, including any compatible substance label 120 correlated with elements of first classified biomarker data 116 utilized in first training set 112 as described above; compatible substance label 120 may be linked to or refer to entries in classified biomarker database 200 to which compatible substance label 120 correspond. Linking may be performed by reference to historical data concerning biomarkers, such as a data entry in classified biomarker database 200 may be determined by reference to a record in an expert knowledge database 204 linking a given compatible substance label 120 to a given category of biomarker data as described above. Entries in compatible substance label 120 database 212 may be associated with one or more categories of compatible substance label 120 as described above, for instance using data stored in and/or extracted from an expert knowledge database 204.

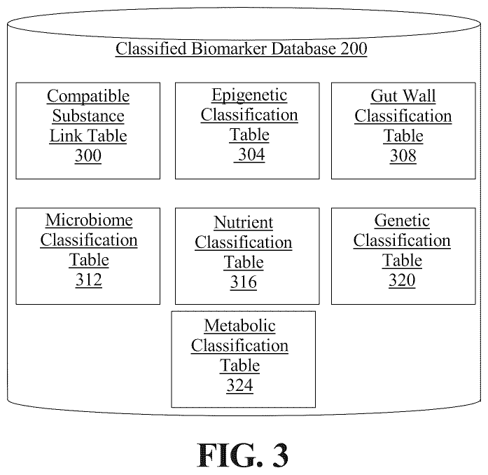

Referring now to FIG. 3, an exemplary embodiment of classified biomarker database 200 is illustrated. Classified biomarker database 200 may include tables listing one or more biomarkers classified according to body dimension. For instance and without limitation, classified biomarker database 200 may include compatible substance link table 300, which may contain information linking compatible substances to classified biomarkers. For instance, and without limitation, biomarker database 200 may include an epigenetic classification table 304 listing biomarkers classified as epigenetic dimension, such as without limitation data describing phenotype biomarkers, behavioral phenotypes, and methylation state of genetic material. As another non-limiting example, biomarker database 200 may include a gut wall classification table 308, which may list biomarkers classified as gut wall dimension, such as without limitation data describing creatinine levels, lactulose levels, zonulin levels, endotoxin lipopolysaccharide (LPS) and the like. As a further non-limiting example, biomarker database 200 may include a microbiome classification table 312, which may list biomarkers classified as microbiome dimension, such as without limitation sequences of microbes found on or within different surfaces of the body as well as data describing current microbe activity. As a further example, also non-limiting, biomarker database 200 may include a nutrient classification table 316, which may list biomarkers classified as nutrient dimension, including without limitation data describing intra and extra cellular concentrations in white and red blood cells of different vitamins, and micronutrients. As a further non-limiting example, classified biomarker database 200 may include genetic classification table 320, which may list biomarkers classified as genetic dimension, such as without limitation data describing partial or entire sequences of genetic material and genetic mutations. As a further non-limiting example, classified biomarker database 200 may include metabolic classification table 324, which may list biomarkers classified as metabolic dimension including for example blood, salivary, hair, skin, urine, and buccal swabs indicating current hormone states, current metabolic rate, and the like. Tables presented above are presented for exemplary purposes only; persons skilled in the art will be aware of various ways in which data may be organized in biomarker database 200 consistently with this disclosure.

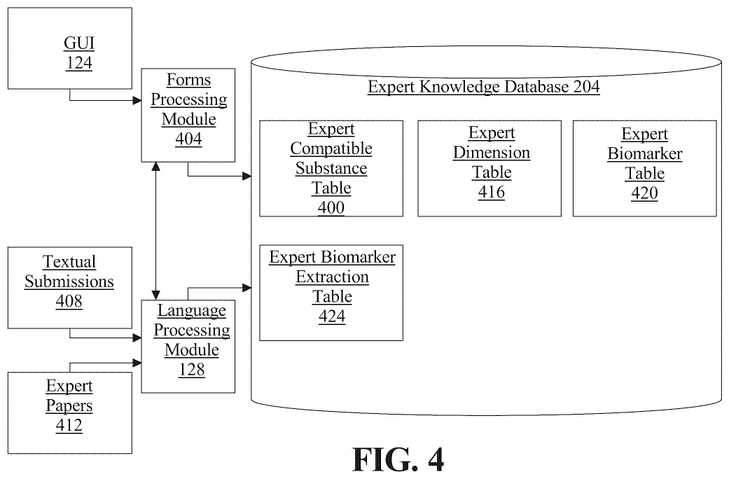

Referring now to FIG. 4, an exemplary embodiment of expert knowledge database 204 is illustrated. One or more database tables in expert knowledge database 204 may include, as a non-limiting example, an expert compatible substance table 400. Expert compatible substance table 400 may be a table relating classified biomarker data as described above to expert compatible substance label 120; for instance, where an expert has entered data relating a compatible substance label 120 to a category of classified biomarker data and/or to an element of classified biomarker data via graphical user interface 124 as described above, one or more rows recording such an entry may be inserted in expert compatible substance table 400. In an embodiment, a forms processing module 404 may sort data entered in a submission via graphical user interface 124 by, for instance, sorting data from entries in the graphical user interface 124 to related categories of data; for instance, data entered in an entry relating in the graphical user interface 124 to a compatible substance label 120 may be sorted into variables and/or data structures for storage of compatible substance label 120, while data entered in an entry relating to a category of classified biomarker data and/or an element thereof may be sorted into variables and/or data structures for the storage of, respectively, categories of classified biomarker data or elements of classified biomarker data. Where data is chosen by an expert from pre-selected entries such as drop-down lists, data may be stored directly; where data is entered in textual form, language processing module 128 may be used to map data to an appropriate existing label, for instance using a vector similarity test or other synonym-sensitive language processing test to map classified biometric data to an existing label. Alternatively or additionally, when a language processing algorithm, such as vector similarity comparison, indicates that an entry is not a synonym of an existing label, language processing module 128 may indicate that entry should be treated as relating to a new label; this may be determined by, e.g., comparison to a threshold number of cosine similarity and/or other geometric measures of vector similarity of the entered text to a nearest existent label, and determination that a degree of similarity falls below the threshold number and/or a degree of dissimilarity falls above the threshold number. Data from expert textual submissions 408, such as accomplished by filling out a paper or PDF form and/or submitting narrative information, may likewise be processed using language processing module 128 114. Data may be extracted from expert papers 412, which may include without limitation publications in medical and/or scientific journals, by language processing module 128 via any suitable process as described herein. Persons skilled in the art, upon reviewing the entirety of this disclosure, will be aware of various additional methods whereby novel terms may be separated from already-classified terms and/or synonyms therefore, as consistent with this disclosure. Expert compatible substance table 400 may include a single table and/or a plurality of tables; plurality of tables may include tables for particular categories of compatible substance label 120 such as an epigenetic table, a gut wall table, a microbiome table, a nutrient table, a genetic table, and a metabolic table (not shown), to name a few non-limiting examples presented for illustrative purposes only.

With continued reference to FIG. 4, one or more database tables in expert knowledge database 204 may include, an expert dimension table 416 may list one or more body dimensions as described by experts, and one or more biomarkers associated with one or more body dimensions. As a further example an expert biomarker table 420 may list one or more biomarkers as described and input by experts and associated dimensions that biomarkers may be classified into. As an additional example, an expert biomarker extraction table 424 may include information pertaining to biological extraction and/or medical test or collection necessary to obtain a particular biomarker, such as for example a tissue sample that may include a urine sample, blood sample, hair sample, cerebrospinal fluid sample, buccal sample, sputum sample, and the like. Tables presented above are presented for exemplary purposes only; persons skilled in the art will be aware of various ways in which data may be organized in expert knowledge database 204 consistently with this disclosure

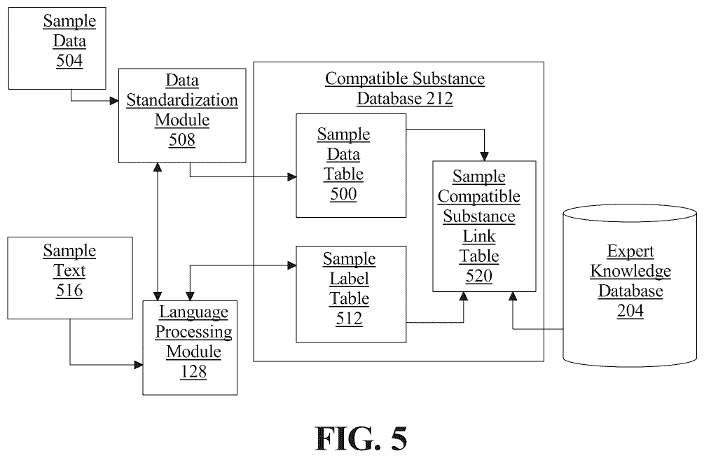

Referring now to FIG. 5, an exemplary embodiment of a compatible substance label 120 database 212 is illustrated. Compatible substance database 212 may, as a non-limiting example, organize data stored in the compatible substance database 212 according to one or more database tables. One or more database tables may be linked to one another by, for instance, common column values. For instance, a common column between two tables of compatible substance database 212 may include an identifier of an expert submission, such as a form entry, textual submission, expert paper, or the like, for instance as defined below; as a result, a query may be able to retrieve all rows from any table pertaining to a given submission or set thereof. Other columns may include any other category usable for organization or subdivision of expert data, including types of expert data, names and/or identifiers of experts submitting the data, times of submission, or the like; persons skilled in the art, upon reviewing the entirety of this disclosure, will be aware of various ways in which expert data from one or more tables may be linked and/or related to expert data in one or more other tables.

Still referring to FIG. 5, one or more database tables in compatible substance database 212 may include, as a non-limiting example, a sample data table 500. Sample data table 500 may be a table listing sample data, along with, for instance, one or more linking columns to link such data to other information stored in compatible substance database 212. In an embodiment, sample data 504 may be acquired, for instance from classified biomarker database 200, in a raw or unsorted form, and may be translated into standard forms, such as standard units of measurement, labels associated with particular physiological data values, or the like; this may be accomplished using a data standardization module 508, which may perform unit conversions. Data standardization module 508 may alternatively or additionally map textual information, such as labels describing values tested for or the like, using language processing module 128 or equivalent components and/or algorithms thereto.

Continuing to refer to FIG. 5, compatible substance database 212 may include a sample label table 512; sample label table 512 may list compatible substance label 120 received with and/or extracted from physiological samples, for instance as received in the form of sample text 516. A language processing module 128 may compare textual information so received to compatible substance labels and/or form new compatible substance label 120 according to any suitable process as described above. Sample compatible substance link table may combine samples with compatible substance label 120, as acquired from sample label table and/or expert knowledge database 204; combination may be performed by listing together in rows or by relating indices or common columns of two or more tables to each other. Tables presented above are presented for exemplary purposes only; persons skilled in the art will be aware of various ways in which data may be organized in expert knowledge database 204 consistently with this disclosure.

Referring now to FIG. 6, an exemplary embodiment of training set database 140 is illustrated which may be implemented in any manner suitable for implementation of classified biomarker database 200. Training set database 140 may contain training sets pertaining to different categories and classifications of information, including training set components which may contain sub-categories of different training sets. One or more database tables contained within training set database 140 table 600 may include without limitation body dimension category table 604; body dimension category table 604 may contain training sets pertaining to different body dimensions correlated to compatible substance label 120. Body dimensions may include for example, epigenetic, gut wall, microbiome, nutrient, genetic, and metabolic and correlated to compatible substance label 120. One or more database tables contained within training set database 140 table 600 may include without limitation tissue category table 608; tissue category table 608 may contain training sets pertaining to different tissue samples that may be analyzed for biomarkers which may be correlated to compatible substance label 120. Tissue may include for example blood, cerebrospinal fluid, urine, blood plasma, synovial fluid, amniotic fluid, lymph, tears, saliva, semen, aqueous humor, vaginal lubrication, bile, mucus, vitreous body, gastric acid, which may be correlated to compatible substance label 120. One or more database tables contained within training set database 140 table 600 may include without limitation medical test table 612; medical test table 612 may contain training sets containing medical tests and medical test results correlated to compatible substance label 120. Medical tests may include any medical test, medical procedure, and/or medical test or procedure results that may be utilized to obtain biomarkers and tissue samples such as for example, an endoscopy procedure utilized to collect a liver tissue sample, or a blood draw collected and analyzed for circulating hormone levels. One or more database tables contained within training set database 140 may include without limitation sensor table 620; sensor table 620 may contain training sets containing sensor data correlated to compatible substance label 120. Sensor data may include any biomarker data that may be obtained from a sensor such as for example, a wearable device that detects a user's sleeping habits or a heart rate monitor contained within a watch. One or more database tables contained within training set database 140 table 600 may include without limitation component category table 624; component category table 624 may contain components or sub-categories of training sets including any of the training sets as described herein. For example, tissue training sets may be broken down in sub-categories such as for example blood tests correlated to compatible substance label 120 and urine tests correlate to compatible substance label 120. Sub-categories may be broken down into further sub-categories such as blood tests that may be further categorized into complete blood count correlated to compatible substance label 120, prothrombin time correlated to compatible substance label 120, metabolic panel correlated to compatible substance label 120, lipid panel correlated to compatible substance label 120, liver panel correlated to compatible substance label 120 and the like. In an embodiment, training sets and/or components of training sets may be categorized and contained within more than one database tables contained within training set database 140 table 600. For instance and without limitation, a training set such as blood glucose test correlated to compatible substance label 120 may be contained within tissue category table 608 and component category table 624. In yet another non-limiting example, a training set such as heart rate correlated to compatible substance label 120 may be categorized and contained within one or more database tables contained within training set database 140 table 600 including for example medical test table 612 and sensor table 620. Tables presented above are presented for exemplary purposes only; persons skilled in the art will be aware of various ways in which data may be organized in training set database 140 consistently with this disclosure.

Referring now to FIG. 7, an exemplary embodiment of machine-learning model database 152 is illustrated which may be implemented in any manner suitable for implementation of classified biomarker database 200. Machine-learning model database 152 may contain machine-learning models categorized and linked to training sets. Machine-learning models may include any of the machine-learning models as described herein. One or more database tables contained within machine-learning model database 152 may include training set link table 704; training set link table 704 may list training sets received with and/or correlated with machine-learning models. Training set link table may combine training sets with machine-learning models, as acquired from expert knowledge database for instance; combination may be performed by listing together in rows or by common columns of two or more tables to each other. One or more database tables contained within machine-learning model database 152 may include body dimension category table; body dimension category table may include machine-learning models correlated to body dimension data, including any of the body dimensions as described herein, including for example epigenetic, gut wall, microbiome, nutrient, genetic, and metabolic. One or more database tables contained within machine-learning model database 152 may include tissue category table 712; tissue category table 712 may include machine-learning models correlated to tissue sample data, including any of the tissue samples as described herein. One or more database tables contained within machine-learning model database 152 may include medical test table 716; medical test table 716 may include machine-learning models correlated to medical test data, including any of the medical tests as described herein. One or more database tables contained within machine-learning model database 152 may include biomarker table 720; biomarker table 720 may include machine-learning models correlated to biomarker data, including any of the biomarkers as described herein. One or more database tables contained within machine-learning model database 152 may include sensor table 724; sensor table 724 may include machine-learning models correlated to sensor data, including any of the sensor data as described herein. One or more database tables contained within machine-learning model database 152 may include component table 728; component table 728 may include machine-learning models correlated to training data component data; including any of the training data components as described herein. Tables presented above are presented for exemplary purposes only; persons skilled in the art will be aware of various ways in which data may be organized in machine-learning model database 152 consistently with this disclosure.

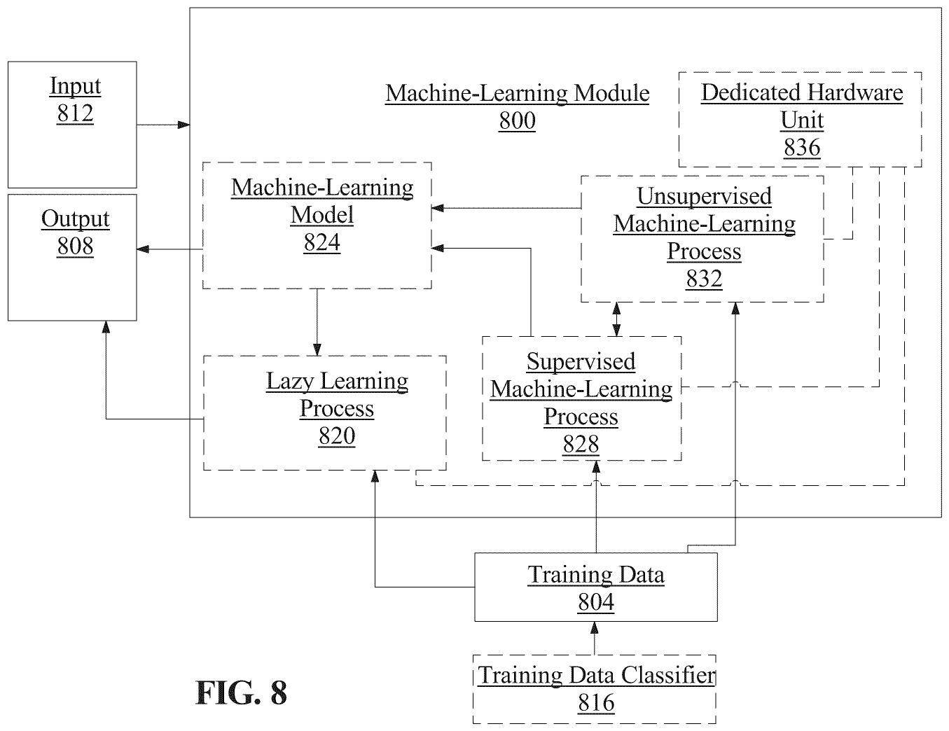

Referring now to FIG. 8, an exemplary embodiment of a machine-learning module 800 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 804 to generate an algorithm instantiated in hardware or software logic, data structures, and/or functions that will be performed by a computing device/module to produce outputs 808 given data provided as inputs 812; this is in contrast to a non-machine learning software program where the commands to be executed are determined in advance by a user and written in a programming language.

Still referring to FIG. 8, “training data,” as used herein, is data containing correlations that a machine-learning process may use to model relationships between two or more categories of data elements. For instance, and without limitation, training data 804 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 804 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 804 according to various correlations; correlations may indicate causative and/or predictive links between categories of data elements, which may be modeled as relationships such as mathematical relationships by machine-learning processes as described in further detail below. Training data 804 may be formatted and/or organized by categories of data elements, for instance by associating data elements with one or more descriptors corresponding to categories of data elements. As a non-limiting example, training data 804 may include data entered in standardized forms by persons or processes, such that entry of a given data element in a given field in a form may be mapped to one or more descriptors of categories. Elements in training data 804 may be linked to descriptors of categories by tags, tokens, or other data elements; for instance, and without limitation, training data 804 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 8, training data 804 may include one or more elements that are not categorized; that is, training data 804 may not be formatted or contain descriptors for some elements of data. Machine-learning algorithms and/or other processes may sort training data 804 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 804 to be made applicable for two or more distinct machine-learning algorithms as described in further detail below. Training data 804 used by machine-learning module 800 may correlate any input data as described in this disclosure to any output data as described in this disclosure. As a non-limiting illustrative example, inputs may include images, and outputs may include body measurements.

Further referring to FIG. 8, training data may be filtered, sorted, and/or selected using one or more supervised and/or unsupervised machine-learning processes and/or models as described in further detail below; such models may include without limitation a training data classifier 816. Training data classifier 816 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 800 may generate a classifier using a classification algorithm, defined as a processes whereby a computing device and/or any module and/or component operating thereon derives a classifier from training data 804. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 816 may classify elements of training data to particular biological sexes.

Still referring to FIG. 8, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 8, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 8, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{θᵢ₌₀aᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 8, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 8, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 8, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 8, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 8, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 8, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 8, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 8, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{I Q R} .$$

Persons skilled in the art, upon reviewing the entirety of this disclosure, will be aware of various alternative or additional approaches that may be used for feature scaling.

Further referring to FIG. 8, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 8, machine-learning module 800 may be configured to perform a lazy-learning process 820 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 804. Heuristic may include selecting some number of highest-ranking associations and/or training data 804 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 8, machine-learning processes as described in this disclosure may be used to generate machine-learning models 824. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 824 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 824 may be generated by creating an artificial neural network, such as a convolutional neural network comprising an input layer of nodes, one or more intermediate layers, and an output layer of nodes. Connections between nodes may be created via the process of “training” the network, in which elements from a training data 804 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 8, machine-learning algorithms may include at least a supervised machine-learning process 828. At least a supervised machine-learning process 828, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include images as described above as inputs, body measurements as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 804. Persons skilled in the art, upon reviewing the entirety of this disclosure, will be aware of various possible variations of at least a supervised machine-learning process 828 that may be used to determine relation between inputs and outputs. Supervised machine-learning processes may include classification algorithms as defined above.

With further reference to FIG. 8, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 8, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 8, machine learning processes may include at least an unsupervised machine-learning processes 832. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 832 may not require a response variable; unsupervised processes 832 may be used to find interesting patterns and/or inferences between variables, to determine a degree of correlation between two or more variables, or the like.

Still referring to FIG. 8, machine-learning module 800 may be designed and configured to create a machine-learning model 824 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 8, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 8, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 8, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 8, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 8, one or more processes or algorithms described above may be performed by at least a dedicated hardware unit 836. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 836 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 836 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 836 to perform one or more operations described herein, such as evaluation of model and/or algorithm outputs, one-time or iterative updates to parameters, coefficients, weights, and/or biases, and/or any other operations such as vector and/or matrix operations as described in this disclosure.

With continued reference to FIG. 8, system 100 may use user feedback to train the machine-learning models and/or classifiers described above. For example, classifier may be trained using past inputs and outputs of classifier. In some embodiments, if user feedback indicates that an output of classifier was “bad,” then that output and the corresponding input may be removed from training data used to train classifier, and/or may be replaced with a value entered by, e.g., another user that represents an ideal output given the input the classifier originally received, permitting use in retraining, and adding to training data; in either case, classifier may be retrained with modified training data as described in further detail below. In some embodiments, training data of classifier may include user feedback.

With continued reference to FIG. 8, in some embodiments, an accuracy score may be calculated for classifier using user feedback. For the purposes of this disclosure, “accuracy score,” is a numerical value concerning the accuracy of a machine-learning model. For example, a plurality of user feedback scores may be averaged to determine an accuracy score. In some embodiments, a cohort accuracy score may be determined for particular cohorts of persons. For example, user feedback for users belonging to a particular cohort of persons may be averaged together to determine the cohort accuracy score for that particular cohort of persons and used as described above. Accuracy score or another score as described above may indicate a degree of retraining needed for a machine-learning model such as a classifier; system 100 may perform a larger number of retraining cycles for a higher number (or lower number, depending on a numerical interpretation used), and/or may collect more training data for such retraining, perform more training cycles, apply a more stringent convergence test such as a test requiring a lower mean squared error, and/or indicate to a user and/or operator that additional training data is needed.

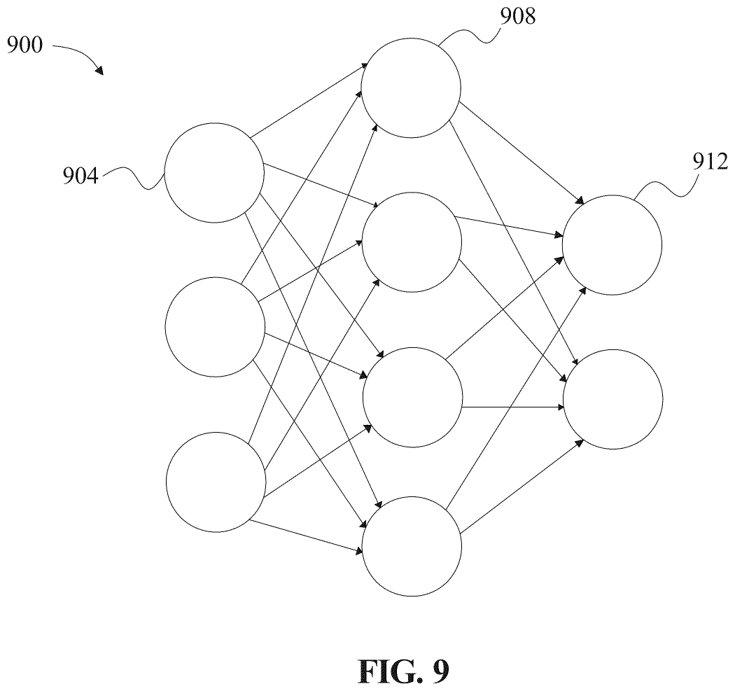

Referring now to FIG. 9, an exemplary embodiment of neural network 900 is illustrated. A neural network 900 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 904, one or more intermediate layers 908, and an output layer of nodes 912. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes.

Referring now to FIG. 10, an exemplary embodiment of a node 1000 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh²(x), a rectified linear unit function such as ƒ(x)=max(0,x), a “leaky” and/or “parametric” rectified linear unit function such as ƒ(x)=max(ax, x) for some a, an exponential linear units function such as

$$f ( x ) = \{ \begin{matrix}x & for x \geq 0 \\ α ( {e}^{x} - 1 ) & for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) & for x < 0 \\ x & for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

With continued reference to FIG. 10, in an embodiment, a machine learning model such as body measurement machine learning model may include a deep neural network (DNN). As used in this disclosure, a “deep neural network” is defined as a neural network with two or more hidden layers. In a non-limiting example, body measurement machine learning model may include a convolutional neural network (CNN). Generation of a body measurement may include training a CNN using example image data and determining a body measurement as a function of an image using a trained CNN. A convolutional neural network may include a neural network in which at least one hidden layer is a convolutional layer that convolves inputs to that layer with a subset of inputs known as a “kernel,” along with one or more additional layers such as pooling layers, fully connected layers, and the like. In some cases, CNN may include, without limitation, a deep neural network (DNN) extension. Mathematical (or convolution) operations performed in the convolutional layer may include convolution of two or more functions, where the kernel may be applied to input data e.g., image data through a sliding window approach. In some cases, convolution operations may enable a processor to detect local/global patterns, edges, textures, and any other features described herein within image data. Spatial features may be passed through one or more activation functions, such as without limitation, Rectified Linear Unit (ReLU), to introduce non-linearities into the processing step of generation of a body measurement. Additionally, or alternatively, CNN may also include one or more pooling layers, wherein each pooling layer is configured to reduce the dimensionality of input data while preserving essential features within the input data. In a non-limiting example, CNN may include one or more pooling layer configured to reduce the spatial dimensions of spatial feature maps by applying downsampling, such as max-pooling or average pooling, to small, non-overlapping regions of one or more features.

Still referring to FIG. 10, CNN may further include one or more fully connected layers configured to combine features extracted by the convolutional and pooling layers as described above. In some cases, one or more fully connected layers may allow for higher-level pattern recognition. In a non-limiting example, one or more fully connected layers may connect every neuron (i.e., node) in its input to every neuron in its output, functioning as a traditional feedforward neural network layer. In some cases, one or more fully connected layers may be used at the end of CNN to perform high-level reasoning and produce the final output such as, without limitation, body measurements. Further, each fully connected layer may be followed by one or more dropout layers configured to prevent overfitting, and one or more normalization layers to stabilize the learning process described herein.

With continued reference to FIG. 10, in an embodiment, training body measurement machine learning model (which may include a CNN) may include selecting a suitable loss function to guide the training process. In a non-limiting example, a loss function that measures the difference between the predicted body measurement and a ground truth structure e.g., example body measurements may be used, such as, without limitation, mean squared error (MSE) or a custom loss function may be designed for one or more embodiments described herein. Additionally, or alternatively, optimization algorithms, such as stochastic gradient descent (SGD), may then be used to adjust the body measurement machine learning model's parameters to minimize such loss. In a further non-limiting embodiment, instead of directly predicting body measurements. Body measurement machine learning model may be trained as a regression model to predict an output in the form of a numeric value. Additionally, CNN may be extended with additional deep learning techniques, such as recurrent neural networks (RNNs) or attention mechanism, to capture additional features and/or data relationships within input data. These extensions may further enhance the accuracy and robustness of the generation of a body measurement.

Still referring to FIG. 10, a “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like. CNN may include, without limitation, a deep neural network (DNN) extension, where a DNN is defined as a neural network with two or more hidden layers.

Still referring to FIG. 10, in some embodiments, a convolutional neural network may learn from images. In non-limiting examples, a convolutional neural network may perform tasks such as classifying images, detecting objects depicted in an image, segmenting an image, and/or processing an image. In some embodiments, a convolutional neural network may operate such that each node in an input layer is only connected to a region of nodes in a hidden layer. In some embodiments, the regions in aggregate may create a feature map from an input layer to the hidden layer. In some embodiments, a convolutional neural network may include a layer in which the weights and biases for all nodes are the same. In some embodiments, this may allow a convolutional neural network to detect a feature, such as an edge, across different locations in an image.

Referring now to FIG. 11, an exemplary embodiment of tissue sample analysis database 1100 is illustrated, which may be which may be implemented in any manner suitable for implementation of classified biomarker database 200. Tissue sample analysis database 1100 may contain information pertaining to tissue sample analysis and results obtained from tissue sample analysis. Tissue sample analysis database 1100 may contain information pertaining to previously recorded tissue samples that may be utilized alone or in combination with biomarker datum to generate compatible element labels. One or more tables contained within tissue sample analysis database 1100 may include training data link table 1104; training data link table 1104 may combine tissue samples with training data sets, as acquired from training set database 140 and expert knowledge database 204. Combination may be performed by listing together in rows or by relating indices or common columns of two or more tables to each other. One or more tables contained within tissue sample analysis database 1100 may include machine-learning model link table 1108; machine-learning link table 1108 may combine tissue samples with machine-learning models, as acquired from machine-learning model database 152 and expert knowledge database 204. Combination may be performed by listing together in rows or by relating indices or common columns of two or more tables to each other. One or more tables contained within tissue sample analysis database 1100 may include blood sample table 1112; blood sample table 1112 may include information describing one or more previous blood samples that a user may have collected and/or had performed. Blood sample may include any kind of blood test or blood analysis such as for example, blood glucose test, calcium blood test, cardiac enzyme test, cholesterol test, c-reactive protein test, serum progesterone level test, serum estradiol level test and the like. One or more database tables contained within tissue sample analysis database 1100 may include saliva sample table 1120; saliva sample table 1120 may include information describing one or more previous saliva samples that a user may have collected and/or had analyzed. Saliva sample may include any kind of salivary test used to detect cortisol levels, hormone levels, gene sequences, gene mutations, heavy metals, iodine levels, and the like. One or more tables contained within tissue sample analysis database 1100 may include urine sample table 1104; urine sample table 1124 may include information describing one or more previous urine samples that a user may have collected and/or had performed. Urine sample may include any kind of urinary test or urinary analysis such as for example to evaluate neurotransmitter levels, iodine levels, heavy metals, hormone levels, ketone levels, absence or presence of bacterial species, absence or presence of fungal species and the like. One or more tables contained within tissue sample analysis database 1100 may include hair sample table 1128; hair sample table 1128 may include information describing one or more previous hair samples that a user may have had analyzed. Hair sample may include any type of hair analysis such as for heavy metal toxicity, bisphenol levels, genetic sequencing, nutrient level evaluation. Other tables contained within tissue sample analysis may include for example, cerebrospinal fluid, blood plasma, synovial fluid, amniotic fluid, lymph, tears, semen, vaginal lubrication, aqueous humor, bile, mucus, vitreous body, gastric acid, muscle biopsy, nervous tissue, epithelial tissue, connective tissue, (not pictured) and the like. Persons skilled in the art, upon reviewing the entirety of this disclosure, will be aware of various forms which may be suitable for use as tissue sample analysis database consistently with this disclosure.

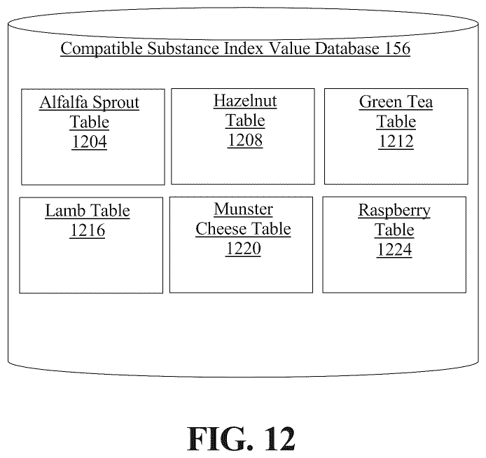

Referring now to FIG. 12, an exemplary embodiment of a compatible substance index value database 156 is illustrated, which may be implemented in any manner suitable for implementation of classified biomarker database 200. Compatible substance index value database 156 may include information describing compatible substance index values for different foods. Compatible substance index value database 156 may be consulted by at least a server 104 when selecting and generating at least a compatible substance instruction set. Compatible substance index value is a value assigned to a compatible substance indicating a degree of compatibility between a first compatible element and a second compatible element for a user with any given biomarker datum. Compatible substance index value may be calculated using any of the methodologies as described above in reference to FIG. 1. Compatible substance index value may contain information allowing for at least a server 104 to select one or more compatible substances as a function of another compatible substance. Compatible substance index value may also allow for at least a server 104 to select compatible substances that may be categorized as belonging to a shared category, such as for example grains or vegetables as described below in more detail in reference to FIG. 11. One or more database tables contained within compatible substance index value database 156 may include alfalfa sprout table 1204; alfalfa sprout table 1204 may include compatible substance index values for alfalfa sprout for any given biomarker datum. For example, alfalfa sprouts may contain a high compatible substance index value for a biomarker that shows low levels of Streptococcus thermophilus while alfalfa sprouts may contain a low compatible substance index value for a biomarker that shows high levels of Lactobacillus lactis. One or more database tables contained within compatible substance index value database 156 may include hazelnut table 1208; hazelnut table 1208 may include compatible substance index values for hazelnuts for any given biomarker datum. For instance and without limitation, hazelnut may have a high compatible substance index value for a biomarker that shows low gastrointestinal levels of Streptococcus and Lactobacillus but may have a low compatible substance index value for a biomarker that shows high blood levels of mold. One or more database tables contained within compatible substance index value database 156 may include green tea table 1212; green tea table 1212 may include compatible substance index values for green tea for any given biomarker datum. For example, green tea may contain a high compatible substance index value for a user who is a high metabolizer of the CYP1A2 gene while green tea may contain a low compatible substance index value for a user who is a slow metabolizer of the CYP1A2 gene. One or more database tables contained within compatible substance index value database 156 may include lamb table 1216; lamb table 1216 may include compatible substance index values for lamb for any given biomarker datum. For example, lamb may have a high compatible substance index value for a biomarker such as a nutrient test showing low blood levels of l-carnitine, while lamb may have a moderate compatible substance index value for a biomarker such as a nutrient test showing normal blood levels of l-carnitine. One or more database tables contained within compatible substance index value database 156 may include Munster cheese table 1220; Munster cheese table 1220 may include compatible substance index values for Munster Cheese for any given biomarker datum. For example, Munster cheese may contain a high compatible substance index value for a user who does not have a mutation of the LCT 2q21 gene that controls lactase production, while Munster cheese may contain a low compatible substance index value for a user who does have a mutation of the LCT 2q21 gene and is unable to produce lactase. One or more database tables contained within compatible substance index value database 156 may include raspberry table 1224; raspberry table 1224 may include compatible substance index values for raspberries for any given biomarker datum. For example, raspberry may contain a low compatible substance index value for a biomarker showing presence of raspberry bushy dwarf virus in a user's gastrointestinal system, while raspberry may contain a high compatible substance index value for a biomarker showing APOE4 gene mutation that recommends blueberries, which may have a high compatible substance index value to be substituted instead for raspberries or recommended together in conjunction with blueberries for a user with the APOE4 gene mutation. Tables contained within compatible substance index value database 156 may include other foods including for example, chestnuts, coffee, cantaloupe melon, pistachios, arugula, bamboo shoots, beet greens, broccoli, burdock root, artichoke, asparagus, beet, bok choy, Brussel sprouts, cabbage, celery (not picture). Persons skilled in the art upon reviewing the entirety of this disclosure, will be aware of various forms which may be suitable for use as compatible substance index value database 156 consistently with this disclosure.

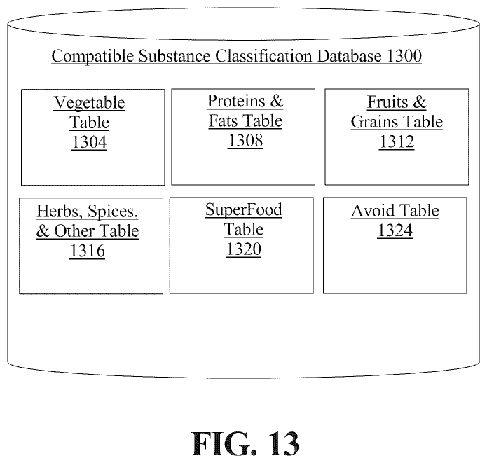

Referring now to FIG. 13, an exemplary embodiment of compatible substance classification database 1300 is illustrated, which may be implemented in any manner suitable for implementation of classified biomarker database 200. Compatible substance classification database 1300 may include information categorizing compatible substances into categories exhibiting shared characteristics. At least a server 104 may consult compatible substance classification database 1300 when generating compatible substance instruction set. In an embodiment, compatible substance instruction set may contain categories that may match categories contained within compatible substance classification database 1300. In an embodiment, at least a server may consult compatible substance classification database 1300 and compatible substance index value database 156 to generate compatible substance instruction set that may contain compatible elements selected based on shared categories contained within compatible substance classification database 1300 and based on compatibility and ability to select a second compatible substance as a function of a first compatible substance based on information contained within compatible substance index value database 156. One or more database tables contained within compatible substance classification database 1300 may include vegetable table 1304; vegetable table 1304 may include all compatible substances classified as vegetables. For example, compatible substances including cauliflower, celery, collard greens, dandelion greens, carrot, cucumber, hard squash, and eggplant may be classified as vegetables. One or more database tables contained within compatible substance classification database 1300 may include proteins and fats table 1308; proteins and fat table 1308 may include all compatible substances classified as proteins and fats. Proteins and fats may include for example, almond milk, avocado oil, grass fed beef, black eyed peas, adzuki beans, anchovy, avocado, black beans bone broth, butter, brazil nuts, chickpeas, chicken, coconut meat, and the like. One or more database tables contained within compatible substance classification database 1300 may include fruits and grains table 1312; fruits and grains table 1312 may include all compatible substances classified as fruits and grains. For example, fruits and grains may include amaranth, apricot, barley, buckwheat, cantaloupe, apple, banana, blackberry, bulgur, cassava, cherry, couscous, currants, dragon fruit, fig, gooseberry, grapes, cranberry, dates, goji berry, grapefruit, huckleberry and the like. One or more database tables contained within compatible substance classification database 1300 may include herbs spices and other table which may include compatible substances classified as herbs, spices, and miscellaneous. Herbs, spices, and other table may include compatible substances such as allspice, bay leaf, cane sugar, caraway seed, celery seed, basil, black pepper, chervil, dill, ginger, honey, cloves, coconut water, herbal tea, horseradish, peppermint, marjoram, molasses, paprika, rosemary and the like. One or more database tables contained within compatible substance classification database 1300 may include superfood table 1320; superfood table 1320 may include all compatible substances classified as superfoods for an individual user. Superfoods may include compatible substances that confer health benefits for a user as a function of a user's biomarker datum. One or more database tables contained within compatible substance classification database 1300 may include avoid table 1324; avoid table 1324 may include compatible substances that do not confer health benefits for a user as a function of a user's biomarker datum and consumption of such compatible substances should be avoided. Persons skilled in the art upon reviewing the entirety of this disclosure, will be aware of various forms which may be suitable for use as compatible substance classification database consistently with this disclosure.

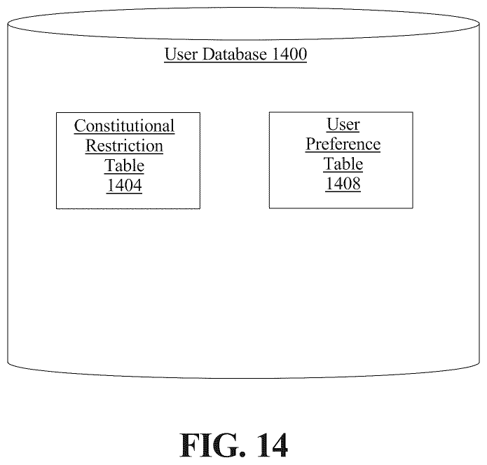

Referring now to FIG. 14, an exemplary embodiment of user database 1400 is illustrated, which may be implemented in any manner suitable for implementation of classified biomarker database 200. In an embodiment, at least a server 104 may consult user database 1400 when generating at least a compatible substance instruction set such as for example, when filtering compatible substance recommendations as a function of user preference or user dietary restrictions. One or more database tables in user database 1400 may include, without limitation, a constitution restriction table 1404; at least a constitutional restriction include information pertaining to a user constitutional restriction which may include any compatible substances that a user chooses not to consume for medical or ethical purposes. For instance and without limitation, constitutional restriction table 1404 may contain information such as a user's preference to eat only vegetarian ingredients. In such an instance, a compatible substance instruction set that contains non-vegetarian compatible substances may be filtered by at least a server 104 to remove such compatible substances from compatible substance instruction set. In yet another non-limiting example, constitutional restriction table 1404 may include information such as a user's self-reported nut allergy, whereby all nut containing compatible substances may be filtered off of a compatible substance instruction set for a user with a previously diagnosed nut allergy. One or more database tables in user database 1400 may include, without limitation, a user preference table 1408; at least a user preference may include information describing a user's preference or aversion to specific compatible substances. For example and without limitation, user preference table 1408 may include information describing a user's aversion to eggs or a user's dislike of tomatoes. In an embodiment, user preference table 1408 may include information describing a user's preference or aversion for categories of compatible substances, such as a user's preference for vegetables or a user's aversion to fruits. Persons skilled in the art upon reviewing the entirety of this disclosure, will be aware of various forms which may be suitable for use as user database consistently with this disclosure.

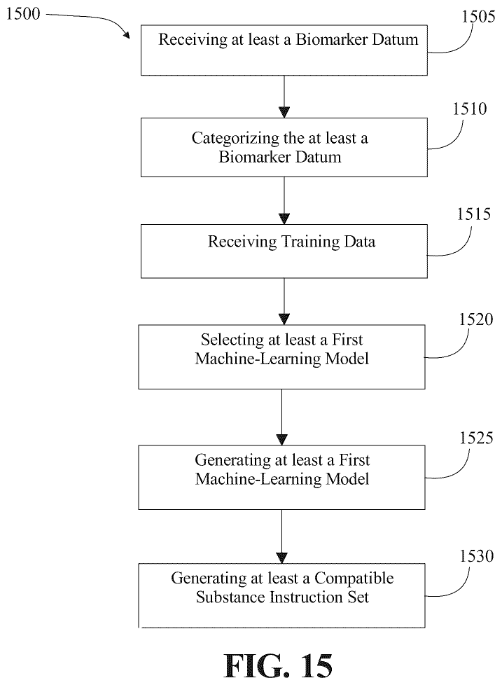

Referring now to FIG. 15, an exemplary embodiment of a method of generating a compatible substance instruction set is illustrated. At step 1505, at least a server receives at least a biomarker datum wherein the at least a biomarker datum contains at least an element of body data correlated to at least a body dimension. Biomarker datum includes any element and/or elements of physiological state data. For example and without limitation, biomarker datum may include a DNA methylation analysis of genes such as FHL2, ZNF518B, GNPNAT1, and HLTF. In yet another non-limiting example, biomarker datum may include a hair test analysis of heavy metals such as arsenic, mercury, cadmium, lead, and aluminum. In yet another non-limiting example, at least a biomarker datum may include a salivary measurement of eosinophil protein x (EPX) or a stool test that contains concentration of a specific strain of bacteria. In an embodiment, at least a biomarker datum may include a tissue sample analysis correlated to at least a body dimension. Tissue sample analysis may include a tissue previously analyzed by a laboratory or medical professional such as a medical doctor for examination. In an embodiment, tissue sample analysis may include comparisons of an extracted tissue sample as compared to reference ranges of normal values or normal findings. For instance and without limitation, tissue sample analysis may include a tissue segment taken from the epithelial lining of a user's gastrointestinal tract and analyzed for microbial content as compared to known reference ranges of microbial contents. In an embodiment, element of body data correlated to at least a body dimension may include at least a datum of user test data containing at least a root system label. User test data may include any of the user test data as described above in reference to FIG. 1. Root system label may include any of the root system labels as described above in reference to FIG. 1. User device may include any of the user devices as described herein. At least a biomarker datum may be received using any network and transmission methodology as described herein.

With continued reference to FIG. 15, at step 1510 at least a biomarker datum is categorized as a function of at least a body dimension to produce at least a classified biomarker datum. Biomarker system classification may include classifying biomarker datums having shared characteristics as related to a dimension of the human body. In an embodiment, biomarker system classification may include classifying at least a biomarker datum as a function of a dimension of the human body. Dimension may include epigenetics, gut wall, microbiome, nutrients, genetics, and metabolism. In an embodiment, classification may include comparing at least a biomarker datum to a classified biomarker datum contained within classified biomarker database 200. For instance and without limitation, at least a biomarker datum containing an extracellular blood level of calcium may be compared to a classified extracellular blood level of calcium contained within classified biomarker database 200 that contains a nutrient classification. In such an instance, at least a biomarker datum that matches a classified biomarker datum contained within classified biomarker database 200 may be utilized to classify at least a biomarker datum. In yet another non-limiting example, at least a biomarker datum such as a salivary hormone level may be compared to a classified salivary hormone level contained within classified biomarker database 200. In such an instance, at least a biomarker datum that matches the classified biomarker datum may then be classified accordingly. In yet another non-limiting example, classifying at least a biomarker may include extracting at least a tissue sample result from at least a biomarker datum and retrieving at least a tissue sample classification label from a database. Tissue sample may include any of the tissue samples as described herein. In an embodiment, at least a biomarker may contain or be linked to at least a tissue sample. For instance and without limitation, at least a biomarker may contain tissue samples or tissue samples may be retrieved from a database such as tissue sample analysis database. In an embodiment, at least a biomarker may be classified as a function of retrieving at least a tissue sample from tissue sample analysis database and classifying at least a biomarker as a function of classification of tissue sample. For instance, at least a biomarker containing a blood sample may be classified as a function of classification label given to blood sample contained within tissue sample analysis database. In yet another non-limiting example, at least a biomarker containing a saliva sample may be classified as a function of a classification label given to a saliva sample contained within saliva sample table located within tissue sample analysis database 1100. In an embodiment, at least a biomarker datum may be classified as a function of language processing module 128. For instance and without limitation, language processing module 128 may extract keywords or text that may accompany at least a biomarker datum. In such an instance, language processing module 128 may extract keywords or trigger words relating to dimensions of the body and utilize such keywords or trigger words to classify the at least a biomarker datum. For instance and without limitation, at least a biomarker datum containing a keyword such as “bacterial strain” and “leaky gut” may be utilized to classify the least a biomarker datum as pertaining to gut wall dimension of body as a function of keywords extracted by language processing module 128. In such an instance, key words extracted by language processing module 128 may be stored within a database, such as for example tissue sample analysis database 1100 or classified biomarker database 200.

With continued reference to FIG. 15, at step 1515 at least a server receives training data. Training data may include any of the training data as described herein. In an embodiment, receiving training data includes receiving a first training set 112 including a plurality of first data entries, each first data entry of the plurality of first data entries including at least a first element of first classified biomarker data 116 and at least a correlated compatible substance label 120. First classified biomarker data 116 may include biomarker data that has been classified to a dimension of the body. For instance and without limitation, biomarker data such as aerobic bacterial cultures, anerobic bacterial cultures, beta-glucuronidase, stool pH, barium enema test results, and stool fat triglyceride levels may be classified as belonging to dimension of the body pertaining to gut wall. In yet another non-limiting example, biomarker data such as extracellular levels of Vitamin A, Vitamin B1, and Vitamin E, and intracellular red blood cell levels of Vitamin D, Vitamin K2, and folate may be classified as belonging to dimension of the body pertaining to nutrients. In an embodiment, biomarker datums may be classified to more than one dimension of the body. For instance and without limitation, biomarker datum such as stool examination for Barnesiella species may be classified as belonging to gut wall dimension and microbiome dimension. In yet another non-limiting example, biomarker datum such as BCMO1 gene that produces enzymes that metabolize and activate Vitamin A may be classified as belonging to genetic dimension and nutrient dimension. Correlated compatible substance label 120 may include any of the correlated compatible substance label 120 as described herein. Compatible substance label 120 may be correlated to at least a classified biomarker data using any of the correlations as described herein, including receiving correlations from experts such as from expert knowledge database. Receiving training data may include receiving at least a first element of classified biomarker datum from at least a constitutional analysis. Constitutional analysis may include any of the constitutional analysis as described above in reference to FIG. 1015. Receiving training data may include receiving at least a first element of classified biomarker datum from at least a tissue sample. Tissue sample may include any of the tissue samples as described above in reference to other figures.

With continued reference to FIG. 15, receiving training data may include receiving a second training set 152. Second training set 132 may include a plurality of second data entries, each second data entry of the plurality of second data entries including at least a second element of second classified biomarker data 136 and at least a correlated compatible substance label 120. Second classified biomarker data 136 may include any biomarker data that has been classified to a second dimension of the body that may be different than the first classified biomarker data 116 received in first training set 112. In an embodiment, at least a server may be configured to receive a plurality of training sets. In an embodiment, receiving a training set may including retrieving a training set from a database, such as for example training set database 140. In an embodiment, receiving a training set may include receiving a component of a training set, such as for example, a sub-set of training data contained within a body dimension training set.

With continued reference to FIG. 15, at step 1520 at least a server selects at least a first machine-learning model 148 as a function of the first training set 112 and the at least a biomarker datum. Selecting at least a machine-learning model may include selecting at least a machine-learning model from machine-learning model database 152. In an embodiment, at least a server 104 may select at least a machine-learning algorithm as a function of the first training set 112. Machine-learning model database 152 may contain information linking machine-learning algorithms to training sets, such as those contained within training set database 140. For instance and without limitation, a training set selected from training set database 140 may be selected and linked to a machine-learning model such as k-nearest neighbor algorithm within machine-learning model database 152. In yet another non-limiting example, a training set selected from training set database 140 may be selected and linked to an unsupervised machine-learning process within machine-learning model database 152. In an embodiment, machine-learning models contained within machine-learning model database 152 may be categorized according to different categories of training sets. For instance, a training set relating body dimension categories to compatible substance label 120 may contain information pertaining to machine-learning algorithms to select for those particular training sets within machine-learning model database 152. In an embodiment, at least a machine-learning model may be selected as a function of the at least a categorized biomarker datum. For instance and without limitation, at least a biomarker datum that is classified as belonging to gut wall body dimension, may be utilized to select at least a machine-learning model that relates to gut wall dimension from machine-learning model database 152 such as by consulting body dimension table 708. In yet another non-limiting example, at least a biomarker datum that is classified as belonging to epigenetic body dimension may be utilized to select at least a machine-learning model that relates to epigenetic body dimension from machine-learning model database 152 such as by consulting body dimension table 708. In an embodiment, body dimension table 708 may be further broken down into categories of body dimensions including for example, epigenetic, gut wall, microbiome, nutrient, genetic, and metabolic. In an embodiment, at least a biomarker may be utilized to select at least a machine-learning algorithm. For instance and without limitation, at least a biomarker may be utilized to select at least a machine-learning model that relates to the at least a biomarker from machine-learning model database 152 such as by consulting information contained within biomarker table 720. In an embodiment, selecting at least a first machine-learning model may include retrieving at least a first machine-learning model from a database as a function of the at least a first element of first classified biomarker datum contained within the first training set.

With continued reference to FIG. 15, at step 1525 at least a server generates at least a first machine-learning model using the first training set wherein the first machine-learning model outputs at least a compatible substance containing at least a compatible substance index value as a function of relating the at least a user biomarker datum to at least a compatible substance using the first training set and the at least a first machine-learning model. Machine-learning models may include any of the machine-learning models as described above in reference to other figures. In an embodiment, this may include generating several machine-learning models. For instance and without limitation, a plurality of biomarkers may be utilized to select several machine-learning models. For example, each biomarker of plurality of biomarkers may select a plurality of machine-learning algorithms that may be utilized to generate at least a compatible substance instruction set. For instance and without limitation, at least a biomarker that contains a blood sample, a urine analysis, and a microbiome sequence may each be utilized to select at least a machine-learning model to generate at least a compatible substance instruction set. In yet another non-limiting example, at least a server may generate an unsupervised machine-learning model followed by a supervised machine-learning model which may include any of the models as described above. In yet another non-limiting example, at least a server may generate an unsupervised machine-learning algorithm followed by a neural network model. This may include any of the machine-learning models as described above in reference to other figures.

With continued reference to FIG. 15, at step 1530 at least a server generates at least a compatible substance instruction set containing at least a compatible substance ranked as a function of the at least a compatible substance index value. Generating compatible substance instruction set may include retrieving at least a compatible substance index value from a database and generating at least a compatible substance instruction set as a function of the at least a compatible substance index value. Compatible substance index value may include any of the compatible substance index values as described above in reference to FIG. 1 and FIG. 10. In an embodiment, at least a compatible substance may be selected and included within compatible substance instruction set as a function of compatible substance index value. For example, a compatible substance containing a high compatible substance index value for a given user's biomarker may be selected and included within compatible substance instruction set. In yet another non-limiting example, a compatible substance containing a low compatible substance index value for a given user's biomarker may not be selected and included within compatible substance instruction set. In an embodiment, a first compatible substance may be selected as a function of a first compatible substance index value and a second compatible substance may be selected as a function of the first compatible substance index value and a second compatible substance index value. For instance and without limitation, a compatible substance index value may provide information as to whether a second compatible substance may be selected as a function of a first compatible substance. For example, a compatible substance index value for blueberries may be utilized and compared to a compatible substance index values for raspberries to determine whether raspberries may be selected and recommended to a user as a function of recommending blueberries to a user. In an embodiment, compatible substance index value may be utilized to generate recommendations based on classifications of compatible substances, such as the classification scheme described above in reference to FIG. 11. For instance, a compatible substance index value for a vegetable such as kale may be utilized and compared to compatible substance index value for a vegetable such as collard greens to determine whether collard greens can be recommended as a function of recommending kale. Compatible substance index value may be evaluated as a function of at least a biomarker datum received from a user client device 108. For instance and without limitation, compatible substance index value may be linked to a user biomarker datum. For example, a first user with a biomarker for a urinary test of bacteria may be linked to a compatible substance index value for the first user with the biomarker result while a second user with the same biomarker for a urinary test of bacteria may be linked to a compatible substance index value for the second user. In an embodiment, compatible substance index value may contain different values for different biomarkers. For example, a compatible substance index value for yellow squash may have a high compatible substance index value for a user with a first genetic mutation but may have a low compatible substance index value for a user with a second genetic mutation or who does not contain the genetic mutation of the first user.

With continued reference to FIG. 15, compatible substance instruction set may contain at least a recommended compatible substance. Recommended compatible substance may include any food recommended for a user. In an embodiment, compatible substance instruction set may be filtered to contain foods that take into account user preference for eating particular foods or dietary eliminations due to food allergies or intolerances. This may be done for instance, such as by consulting user database 1200. In an embodiment, compatible substance instruction set may organize recommended compatible substances into categories such as for example utilizing the categorization scheme as described above in reference to FIG. 13 in compatible substance classification database 1300. For example, compatible substance instruction set may contain categories listing recommended compatible substances under each category such as for example a vegetable category that may include kale, cauliflower, and beets and a fruits and grain category that may contain watermelon, buckwheat, and couscous.

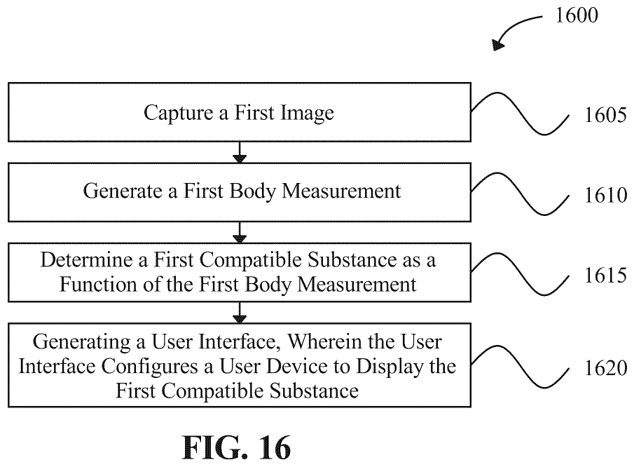

Referring now to FIG. 16, an exemplary embodiment of a method 1600 of determining a compatible substance is illustrated. One or more steps if method 1600 may be implemented, without limitation, as described with reference to other figures. One or more steps of method 1600 may be implemented, without limitation, using at least a processor.

Still referring to FIG. 16, in some embodiments, method 1600 may include capturing a first image 1605. In some embodiments, first image may be captured using a camera.

Still referring to FIG. 16, in some embodiments, method 1600 may include generating a first body measurement 1610. In some embodiments, generating a first body measurement may include training a body measurement machine learning model on a training dataset including a plurality of example images correlated to a plurality of example body measurements; and generating the first body measurement as a function of the first image using the trained body measurement machine learning model. In some embodiments, the body measurement comprises a metric selected from the list consisting of visceral fat content, height, weight, body mass index, lean body mass, body water percentage, and bone density.

Still referring to FIG. 16, in some embodiments, method 1600 may include determining a first compatible substance as a function of the first body measurement 1615.

Still referring to FIG. 16, in some embodiments, method 1600 may include generating a user interface, wherein the user interface configures a user device to display the first compatible substance.

Still referring to FIG. 16, in some embodiments, method 1600 may further include receiving a subject health datum; using a camera, capturing a second image as a function of the subject health datum; generating a second body measurement as a function of the second image using the trained body measurement machine learning model; determining a second compatible substance as a function of the second body measurement; and using the user interface, displaying the second compatible substance. In some embodiments, method 1600 may further include identifying a body measurement impact ingredient; generating a nutrient plan as a function of the body measurement impact ingredient; and using the user interface, displaying the nutrient plan.

Still referring to FIG. 16, in some embodiments, method 1600 may further include generating a first digital avatar as a function of the first body measurement; and using the user interface, displaying the first digital avatar. In some embodiments, the first digital avatar comprises a 3 dimensional avatar. In some embodiments, method 1600 may further include using the user interface, receiving a body measurement adjustment datum; generating a second body measurement as a function of the body measurement adjustment datum; generating a second digital avatar as a function of the second body measurement; and using the user interface, displaying the second digital avatar. In some embodiments, method 1600 may further include generating a health improvement body measurement estimation; generating a second digital avatar as a function of the health improvement body measurement estimation; and using the user interface, displaying the second digital avatar.

Still referring to FIG. 16, in some embodiments, the first body measurement comprises a body fat distribution. In some embodiments, method 1600 may further include determining a medical condition risk datum as a function of the body fat distribution; and using the user interface, displaying the medical condition risk datum.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

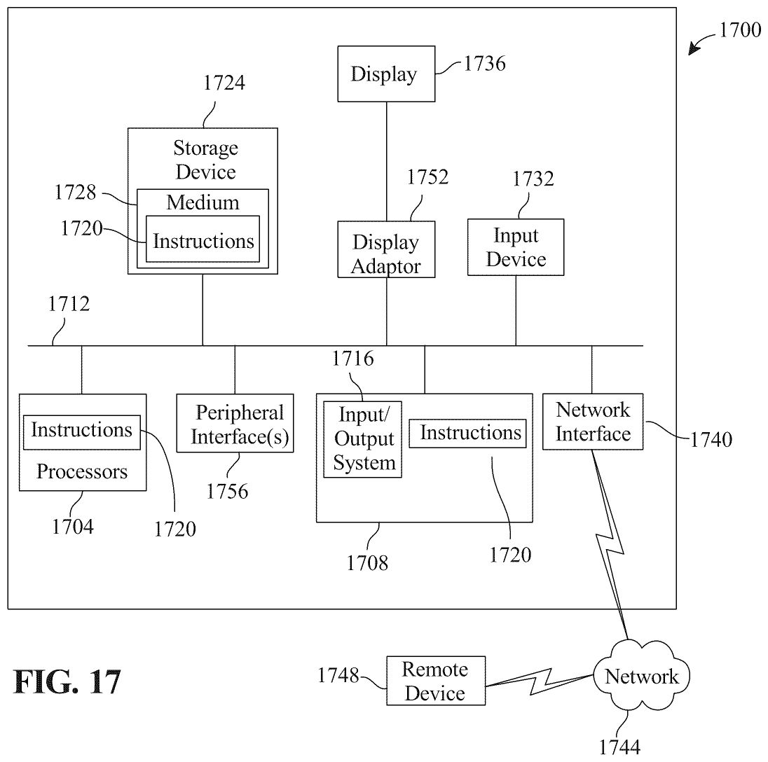

FIG. 17 shows a diagrammatic representation of one embodiment of a computing device in the exemplary form of a computer system 1700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1700 includes a processor 1704 and a memory 1708 that communicate with each other, and with other components, via a bus 1712. Bus 1712 may include any of several types of bus structures including, but not limited to, a memory bus, a memory controller, a peripheral bus, a local bus, and any combinations thereof, using any of a variety of bus architectures.

Processor 1704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1704 may be organized according to Von Neumann and/or Harvard architecture as a non-limiting example. Processor 1704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1708 may include various components (e.g., machine-readable media) including, but not limited to, a random-access memory component, a read only component, and any combinations thereof. In one example, a basic input/output system 1716 (BIOS), including basic routines that help to transfer information between elements within computer system 1700, such as during start-up, may be stored in memory 1708. Memory 1708 may also include (e.g., stored on one or more machine-readable media) instructions (e.g., software) 1720 embodying any one or more of the aspects and/or methodologies of the present disclosure. In another example, memory 1708 may further include any number of program modules including, but not limited to, an operating system, one or more application programs, other program modules, program data, and any combinations thereof.

Computer system 1700 may also include a storage device 1724. Examples of a storage device (e.g., storage device 1724) include, but are not limited to, a hard disk drive, a magnetic disk drive, an optical disc drive in combination with an optical medium, a solid-state memory device, and any combinations thereof. Storage device 1724 may be connected to bus 1712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1724 (or one or more components thereof) may be removably interfaced with computer system 1700 (e.g., via an external port connector (not shown)). Particularly, storage device 1724 and an associated machine-readable medium 1728 may provide nonvolatile and/or volatile storage of machine-readable instructions, data structures, program modules, and/or other data for computer system 1700. In one example, software 1720 may reside, completely or partially, within machine-readable medium 1728. In another example, software 1720 may reside, completely or partially, within processor 1704.

Computer system 1700 may also include an input device 1732. In one example, a user of computer system 1700 may enter commands and/or other information into computer system 1700 via input device 1732. Examples of an input device 1732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1732 may be interfaced to bus 1712 via any of a variety of interfaces (not shown) including, but not limited to, a serial interface, a parallel interface, a game port, a USB interface, a FIREWIRE interface, a direct interface to bus 1712, and any combinations thereof. Input device 1732 may include a touch screen interface that may be a part of or separate from display device 1736, discussed further below. Input device 1732 may be utilized as a user selection device for selecting one or more graphical representations in a graphical interface as described above.

A user may also input commands and/or other information to computer system 1700 via storage device 1724 (e.g., a removable disk drive, a flash drive, etc.) and/or network interface device 1740. A network interface device, such as network interface device 1740, may be utilized for connecting computer system 1700 to one or more of a variety of networks, such as network 1744, and one or more remote devices 1748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1744, may employ a wired and/or a wireless mode of communication. In general, any network topology may be used. Information (e.g., data, software 1720, etc.) may be communicated to and/or from computer system 1700 via network interface device 1740.

Computer system 1700 may further include a video display adapter 1752 for communicating a displayable image to a display device, such as display device 1736. Examples of a display device include, but are not limited to, a liquid crystal display (LCD), a cathode ray tube (CRT), a plasma display, a light emitting diode (LED) display, and any combinations thereof. Display adapter 1752 and display device 1736 may be utilized in combination with processor 1704 to provide graphical representations of aspects of the present disclosure. In addition to a display device, computer system 1700 may include one or more other peripheral output devices including, but not limited to, an audio speaker, a printer, and any combinations thereof. Such peripheral output devices may be connected to bus 1712 via a peripheral interface 1756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for determining a compatible substance, the system comprising a computing device configured to:
using a camera, capture a first image;
generate a first body measurement by:
training a body measurement machine learning model on a training dataset including a plurality of example images correlated to a plurality of example body measurements; and
generating the first body measurement as a function of the first image using the trained body measurement machine learning model;
determine a first compatible substance as a function of the first body measurement; and
generate a user interface, wherein the user interface configures a user device to display the first compatible substance.

2. The system of claim 1, wherein the body measurement comprises a body mass metric.

3. The system of claim 1, wherein the computing device is further configured to:
receive a subject health datum;
capture a second image as a function of the subject health datum;
generate a second body measurement as a function of the second image using the trained body measurement machine learning model;
determine a second compatible substance as a function of the second body measurement; and
using the user interface, configure the user device to display the second compatible substance.

4. The system of claim 1, wherein the computing device is further configured to:
identify a body measurement impact ingredient;
generate a nutrient plan as a function of the body measurement impact ingredient; and
using the user interface, configure the user device to display the nutrient plan.

5. The system of claim 1, wherein the computing device is further configured to:
generate a first digital avatar as a function of the first body measurement; and
using the user interface, configure the user device to display the first digital avatar.

6. The system of claim 5, wherein the first digital avatar comprises a three-dimensional avatar.

7. The system of claim 5, wherein the computing device is further configured to:
using the user interface, receive a body measurement adjustment datum;
generate a second body measurement as a function of the body measurement adjustment datum;
generate a second digital avatar as a function of the second body measurement; and
using the user interface, configure the user device to display the second digital avatar.

8. The system of claim 5, wherein the computing device is further configured to:
generate a health improvement body measurement estimation;
generate a second digital avatar as a function of the health improvement body measurement estimation; and
using the user interface, configure the user device to display the second digital avatar.

9. The system of claim 1, wherein the first body measurement comprises a body fat distribution.

10. The system of claim 9, wherein the computing device is further configured to:
determine a medical condition risk datum as a function of the body fat distribution; and
using the user interface, configure the user device to display the medical condition risk datum.

11. A method of determining a compatible substance, the method comprising:
using a camera and at least a processor, capturing a first image;
using the at least a processor, generating a first body measurement by:
training a body measurement machine learning model on a training dataset including a plurality of example images correlated to a plurality of example body measurements; and
generating the first body measurement as a function of the first image using the trained body measurement machine learning model;
using the at least a processor, determining a first compatible substance as a function of the first body measurement; and
using the at least a processor, generating a user interface, wherein the user interface configures a user device to display the first compatible substance.

12. The method of claim 11, wherein the body measurement comprises a body mass metric.

13. The method of claim 11, wherein the method further comprises:
using the at least a processor, receiving a subject health datum;
using the at least a processor, capturing a second image as a function of the subject health datum;
using the at least a processor, generating a second body measurement as a function of the second image using the trained body measurement machine learning model;
using the at least a processor, determining a second compatible substance as a function of the second body measurement; and
using the at least a processor and the user interface, configuring the user device to display the second compatible substance.

14. The method of claim 11, wherein the method further comprises:
using the at least a processor, identifying a body measurement impact ingredient;
using the at least a processor, generating a nutrient plan as a function of the body measurement impact ingredient; and
using the user interface and the at least a processor, configuring the user device to display the nutrient plan.

15. The method of claim 11, wherein the method further comprises:
using the at least a processor, generating a first digital avatar as a function of the first body measurement; and
using the user interface and the at least a processor, configuring the user device to display the first digital avatar.

16. The method of claim 15, wherein the first digital avatar comprises a three-dimensional avatar.

17. The method of claim 15, wherein the method further comprises:
using the user interface and the at least a processor, receiving a body measurement adjustment datum;
using the at least a processor, generating a second body measurement as a function of the body measurement adjustment datum;
using the at least a processor, generating a second digital avatar as a function of the second body measurement; and
using the user interface and the at least a processor, configuring the user device to display the second digital avatar.

18. The method of claim 15, wherein the method further comprises:
using the at least a processor, generating a health improvement body measurement estimation;
using the at least a processor, generating a second digital avatar as a function of the health improvement body measurement estimation; and
using the user interface and the at least a processor, configuring the user device to display the second digital avatar.

19. The method of claim 11, wherein the first body measurement comprises a body fat distribution.

20. The method of claim 19, wherein the method further comprises:
using the at least a processor, determining a medical condition risk datum as a function of the body fat distribution; and
using the user interface and the at least a processor, configuring the user device to display the medical condition risk datum.
